(19) United States
(12) Patent Application Publication
(10) Pub. No.: US 2003/0157091 A1
(43) Pub. Date: Aug. 21, 2003

(54) Multi-functional proteins

(75) Inventor: Henricus Renerus Jacobus Mattheus Hoogenboom, Maastricht (NL)

Correspondence Address: FISH & RICHARDSON PC, 225 FRANKLIN ST, BOSTON, MA, 02110, US

(73) Assignee: DYAX CORPORATION

(21) Appl. No.: 10/364,815

(22) Filed: Feb. 11, 2003

Related U.S. Application Data
(60) Provisional application No. 60/357,294, filed on Feb. 14, 2002.

(51) Int. Cl. G01N 33/53; A61K 39/395; C07K 16/18

(52) U.S. Cl. 424/130.1; 530/388.1; 435/7.1

(57) ABSTRACT

Disclosed are compositions and methods to generate functional target-binding proteins from at least two separate polypeptide chains, one including the target-binding domain, the other including an effector domain. For example, the two separate chains are reconstituted as a functional protein by a non-covalent binding interaction mediated by an interaction sequence or by intein-mediated ligation.

### CROSS-REFERENCE TO RELATED APPLICATIONS

This application claims priority to U.S. application Ser. No. 60/357,294, filed on Feb. 14, 2002, the contents of which are incorporated by reference in their entirety.

### BACKGROUND

This application relates to multi-functional proteins. An example of a multi-functional protein is a bi-functional protein that includes a target-binding function, a secondary function such as an effector function, and even, in some cases, additional functions.

Antibodies are at least bifunctional. Antibodies have a versatile polypeptide scaffold that can be adapted to specifically bind any of a vast array of compounds. Natural processes generate a diverse repertoire of antibodies. The repertoire includes antibodies with different amino acid sequences in the N-terminal domains, termed variable domains. Most variation is generated in hypervariable regions or complementarity determining regions (CDR's) within the variable domains. These varied determinants are used by antibodies to specifically bind targets.

In addition to binding targets, antibodies can have a number of effector functions.

One effector function is the recruitment of proteins of the complement cascade. C1q, the first protein effector recognizes aggregated immunoglobulins by binding to their constant domain (Fc). C1q recruits other members of the complement cascade which can form a membrane attachment complex to lyse cells and can release anaphylatoxins to trigger mast cells to increase vascular permeability.

Another effector function is antibody-dependent cell-mediated cytotoxicity (ADCC). Antibodies that bind to an antigen attached to a target cell can recruit leukocytes such as neutrophils, eosinophils, mononuclear phagocytes, and natural killer (NK) cells. These leukocytes express a class of receptors for the Fc region of antibodies, termed the FcγRs. For example, NK cells, the predominant ADCC mediator, expresses FcγRIII on its surface. When the receptor is occupied, the NK cell is triggered to secrete cytokines and to exocytose granules that include pore-forming proteins, cytotoxins, proteases, and proteoglycans. These events kill the antibody-bound target cell.

The properties of antibodies are being exploited in order to design agents that bind to human target molecules, so-called, “self-antigens.” For example, a number of monospecific antibodies have been approved as human therapeutics. These include Orthoclone OKT3, which targets CD3 antigen; ReoPro, which targets GP IIb/IIIa; Rituxan, which targets CD20; Zenapax and Simulect, which target interleukin-2 receptors; Herceptin, which targets the HER2-receptor; Remicade, which targets tumor necrosis factor; Synagis, which targets the F protein of respiratory syncytial virus; Mylotarg, which targets CD33; and Campath, which targets CD52 (see, e.g., Ezzell (2001) Scientific American October 2001, pages 36-41; Garber (2001) Nat. Biotechnol. 19:184-185).

Recombinant nucleic acid technology has enable the cloning and manipulation of nucleic acid sequences that encode antibodies and antibody variants. For example, antibody fragments can be expressed in bacterial cells and on the surface of bacteriophages. Bacteriophages can be used to display antibody (see, e.g., U.S. Pat. No. 5,223,409; WO 92/18619; PCT WO 91/17271; PCT WO 92/20791; PCT WO 92/15679; PCT WO 93/01288; PCT WO 92/01047; PCT WO 92/09690; PCT WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Hoogenboom et al. (1991) Nuc Acid Res 19:4133-4137; and Barbas et al. (1991) Proc. Natl. Acad. Sci. USA 88:7978-7982). These prokaryotic expression systems (e.g., secretion from bacterial cells and phage display) are very efficient for screening the binding properties of antigen-binding domains.

One challenge for assaying the biological properties of antigen-binding domains identified in a prokaryotic screen is testing effector functions that depend on modifications that are unique to eukaryotic systems, e.g., glycosylation. Another challenge for assaying these binding domains is the finding that some antigen-binding domains have altered binding properties when expressed in a eukaryotic system. A common approach is to redone the nucleic acid sequences that encode the antigen-binding domain from a prokaryotic expression vector into a eukaryotic expression vector. The antigen-binding domain is then expressed and purified in the eukaryotic system for binding and functional assays.

### SUMMARY

The inventors have discovered that target-binding and effector functions can be provided by separate polypeptide chains that are subsequently joined, e.g., by a covalent or non-covalent interaction.

In one aspect, the invention features an artificial protein compound that includes: a first polypeptide that includes at least a part of a target-binding sequence and a first interaction sequence; and a second polypeptide that includes the second interaction sequence and at least a part of an effector sequence. The first interaction sequence can interact (e.g., bind) with the second interaction sequence. Further, the effector sequence is has one or more of the following properties: a) binds (e.g., specifically binds) to a surface of a cell, b) is functional in an extracellular environment, and c) is a detectable label (i.e. other than being antigenic) (e.g., generates a signal).

In one embodiment, the target-binding sequence does not include an immunoglobulin domain. For example, the target-binding sequence can include a cytokine, a peptide hormone, or a fragment thereof. In a related example, the target-binding sequence includes a naturally occurring extracellular domain, e.g., a domain that includes a disulfide bond.

In another embodiment, the target-binding sequence includes an immunoglobulin domain, e.g., an immunoglobulin variable domain. The target-binding sequence can include an antigen-binding domain. For example, the first polypeptide can include a VH and/or VL domain. Typically the first polypeptide is at least a component of the antigen-binding domain, e.g., in conjunction with a third polypeptide.. The antigen-binding domain can include the first immunoglobulin domain and a second immunoglobulin domain. The second immunoglobulin domain can be a component of the third polypeptide. The first and second immunoglobulin domains are generally variable domains. For example, the first immunoglobulin can be VH and the second immunoglobulin domain can be VL, or vice versa. In one embodiment, the first polypeptide includes both the VH and VL domain, e.g., a scFv. In another embodiment which includes the third polypeptide, the first polypeptide further includes a CH1 domain and the third polypeptide further includes a CL domain. The first and third polypeptide can be covalently linked by a disulfide bond. The first immunoglobulin variable domain can include one or more synthetic CDRs, or naturally-occurring CDRs, e.g., a germline CDR and/or a somatic mutant thereof. In one embodiment, one or more of the CDRs is a human CDR, e.g., CDR3 is a human CDR. The first immunoglobulin variable domain can include one or more human framework regions.

In another embodiment, the target-binding sequence is synthetic or includes a synthetic region. For example, the synthetic region can be about 6 to 30 amino acids, or longer. In one embodiment, the synthetic region includes a cysteine loop of about 4 to 15 amino acids. In still another embodiment, the target-binding sequence includes a modified scaffold domain. Further, the target-binding sequence can be a region of a naturally-occurring protein, e.g., a region of a mammalian ectodomain.

In one embodiment, the target-binding sequence is less than 50, 30, 20, or 10 kDa or less than 100, 50, or 30 amino acids. In another embodiment, the target-bindingsequence is at least 10, 20, 50 kDa or at least 30, 50, 150, 200 amino acids.

In one embodiment, the target-binding sequence and/or the effector sequence is not antigenic or immuno-reactive in humans. The target-binding sequence and/or effector sequence can include a human sequence or a modified human sequence.

The effector sequence can include, e.g., a domain of an extracellular protein or an extracellular portion of a naturally-occurring protein. The effector sequence can include one or more polypeptide chains, of which one (or more) is a component of the second polypeptide. For example, the effector sequence can include an immunoglobulin effector sequence (e.g., a domain that includes CH2) or a non-immunoglobulin effector sequence.

In an embodiment, the effector sequence is glycosylated. For example, the second polypeptide may be synthesized in a eukaryotic cell, e.g., a mammalian cell, e.g., a mammalian culture cell or in vivo. Further, the first polypeptide may be synthesized in vitro or in a bacterial cell. The first polypeptide can also, of course, be synthesized in a mammalian cell, and likewise the second polypeptide may be synthesized in a bacterial cell or in vitro.

In an embodiment, the effector sequence includes an Fc domain, e.g., CH2 and CH3 domains, e.g., IgG CH2 and IgG CH3 domains. The Fc domain can be glycosylated on at least an asparagine corresponding to asparagine 297 of CH2 (Kabat numbering). The effector sequence can be an Fc domain mutant, e.g., an asymmetric Fc domain and/or a modified specificity Fc domain.

The first and/or second polypeptide can include a flexible region that spaces the interaction sequence from the target-binding sequence or the effector sequence. In one embodiment, the flexible region includes an immunoglobulin hinge domain. The effector sequence can be N-terminal or C-terminal to the second interaction sequence.

In an embodiment, the effector sequence is a non-immunoglobulin effector sequence. The effector sequence, for example, can be an extracellular domain, or at least functional in the extracellular milieu.

Some effector sequences can bind to a cell surface, e.g., they recognize a cell surface receptor. Some effector sequences can elicit a cytotoxic effect. For example, the effector sequence can include a toxin.

In an embodiment, the effector sequence includes a signal effector, e.g., a non-peptide label that is covalently attached to the second polypeptide. For example, the signal effector may be a contrast agent, e.g., an NMR contrast agent. For another example, the signal effector is a fluorescent protein.

In an embodiment, the effector sequence is less than 50, 30, 20, or 10 kDa or less than 150, 50, or 30 amino acids. In another embodiment, the effector sequence is at least 10, 20, 50 kDa or at least 30, 50, 150, 200 amino acids.

The first and second interaction sequences can be complementary heterodimerization sequences. For example, the first and second interaction sequences can be segments of single folded unit. In another example, the first and second interaction sequences are components of a coiled-coil. Such sequences can include a heptad repeat (typically at least 3, 4, or 5 repeats). They can be leucine zippers, e.g., the leucine zippers of fos and jun. They can have an amino acid sequence with fewer than 7, 4, 3, 2, or 1 substitutions relative to SEQ ID NOS: 1 to 8.

The first and second polypeptides can each include a cysteine that forms a disulfide bond with the corresponding cysteine on the other polypeptide, e.g., when the first and second interaction sequences interact.

The first and/or second polypeptide can further include a purification tag (the same or different tag). The protein can also include a non-peptide conjugate.

In an embodiment, the first polypeptide includes a multimer of interaction sequences. For example, the multimer can include two, three, four, five, eight or more repeated units. One or more (e.g., at least two, three, up to and including all) of which is the first interaction sequence. Each of the first interaction sequences can be bound by a replicate of the second polypeptide, e.g., such that one or more replicates of the second polypeptide are components of the protein. Likewise, the second polypeptide can include a multimer of interaction sequences, one or more of which is the second interaction sequence.

In one embodiment, the protein includes two first polypeptides and two second polypeptides. For example, the two second polypeptides can form a homodimer as well as interacting with the respective first polypeptides.

The invention also features nucleic acids that encode the afore-mentioned polypeptides, and kits that include the nucleic acids. For example, an isolated, artificial nucleic acid that includes a sequence encoding a polypeptide that includes an interaction sequence (e.g., a heterodimerization sequence) and an effector sequence is provided. The interaction sequence is heterologous to the effector sequence. In an embodiment, the heterodimerization sequence and the effector sequence are non-overlapping. The nucleic acid can include a human nucleic acid sequence or a modified nucleic acid human sequence.

In another aspect, the invention features a protein that includes a first and a second polypeptide. The first polypeptide includes a first immunoglobulin domain and a first interaction sequence. The second polypeptide includes a second interaction sequence and an effector sequence (or at least a component thereof). Typically, the second polypeptide does not include a functional immunoglobulin variable domain. The first interaction sequence specifically recognizes (e.g., binds) the second interaction sequence.

The first polypeptide can include a VH and/or VL domain. Typically the first polypeptide is at least a component of an antigen-binding domain, e.g., in conjunction with a third polypeptide. The antigen-binding domain can include the first immunoglobulin domain and a second immunoglobulin domain. The second immunoglobulin domain can be a component of the third polypeptide. The first and second immunoglobulin domains are generally variable domains. For example, the first immunoglobulin can be VH and the second immunoglobulin domain can be VL, or vice versa.

In an embodiment, the first polypeptide includes both the VH and VL domain, e.g., a scFv.

In another embodiment which includes the third polypeptide, the first polypeptide further includes a CH1 domain and the third polypeptide further includes a CL domain. The first and third polypeptide can be covalently linked by a disulfide bond.

The first immunoglobulin variable domain can include one or more synthetic CDRs, or naturally-occurring CDRs, e.g., a germline CDR and/or a somatic mutant thereof. In one embodiment, one or more of the CDRs is a human CDR, e.g., CDR3 is a human CDR. The first immunoglobulin variable domain can include one or more human framework regions.

In an embodiment, the protein includes two first polypeptides and two second polypeptides. For example, the two second polypeptides can form a homodimer as well as interacting with the respective first polypeptides.

The effector sequence can, for example, have one or more of the following properties: a) binding (e.g., specifically binding) to a surface of a cell, b) functionality in an extracellular environment, and c) detectability (i.e. other than being antigenic) (e.g., generates a signal). The effector sequence can include a human sequence or a modified human sequence.

In an embodiment, the effector sequence is glycosylated. For example, the second polypeptide may be synthesized in a eukaryotic cell, e.g., a mammalian cell, e.g., a mammalian culture cell or in vivo. Further, the first polypeptide may be synthesized in vitro or in a bacterial cell. The first polypeptide can also, of course, be synthesized in a mammalian cell, and likewise the second polypeptide may be synthesized in a bacterial cell or in vitro.

In an embodiment, the effector sequence includes an Fe domain, e.g., CH2 and CH3 domains, e.g., IgG CH2 and IgG CH3 domains. The Fe domain can be glycosylated on at least an asparagine corresponding to asparagine 297 of CH2 (Kabat numbering). The effector sequence can be an Fc domain mutant, e.g., an asymmetric Fc domain and/or a modified specificity Fc domain.

In an embodiment, the effector sequence is a non-immunoglobulin effector sequence. The effector sequence, for example, can be an extracellular domain, or at least functional in the extracellular milieu.

Some effector sequences can bind to a cell surface, e.g., they recognize a cell surface receptor. Some effector sequences can elicit a cytotoxic effect. For example, the effector sequence can include a toxin. In another example, the effector sequences recruit a cytotoxic cell.

In an embodiment, the effector sequence includes a signal effector, e.g., a non-peptide label that is covalently attached to the second polypeptide. For example, the signal effector may be a contrast agent, e.g., an NMR contrast agent. For another example, the signal effector is a fluorescent protein.

In an embodiment, the effector sequence is less than 50, 30, 20, or 10 kDa or less than 150, 50, or 30 amino acids. In another embodiment, the effector sequence is at least 10, 20, 50 kDa or at least 30, 50, 150, 200 amino acids.

The first and/or second polypeptide can include a flexible region that spaces the interaction sequence from the immunoglobulin domain or the effector sequence. In one embodiment, the flexible region includes an immunoglobulin hinge domain. The effector sequence can be N-terminal or C-terminal to the second interaction sequence.

The first and second interaction sequences can be complementary heterodimerization sequences. For example, the first and second interaction sequences can be segments of single folded unit. In another example, the first and second interaction sequences are components of a coiled-coil. Such sequences can include a heptad repeat (typically at least 3, 4, or 5 repeats). They can be leucine zippers, e.g., the leucine zippers of fos and jun. They can have an amino acid sequence with fewer than 7, 4, 3, 2, or 1 substitutions relative to SEQ ID NOS: 1 to 8.

The first and second polypeptides can each include a cysteine that forms a disulfide bond with the corresponding cysteine on the other polypeptide, e.g., when the first and second interaction sequences interact.

The first and/or second polypeptide can further include a purification tag (the same or different tag). The protein can also include a non-peptide conjugate.

In an embodiment, the first polypeptide includes a multimer of interaction sequences. For example, the multimer can include two, three, four, five, eight or more repeated units. One or more (e.g., at least two, three, up to and including all) of which is the first interaction sequence. Each of the first interaction sequences can be bound by a replicate of the second polypeptide, e.g., such that one or more replicates of the second polypeptide are components of the protein. Likewise, the second polypeptide can include a multimer of interaction sequences, one or more of which is the second interaction sequence.

The invention also features nucleic acids that encode the afore-mentioned polypeptides, and kits that include the nucleic acids.

In still another aspect, the invention features an artificial protein complex that includes a first, second, and third protein. The first protein includes a first and second heterodimerization sequence and an effector sequence. The second protein includes a third heterodimerization sequence and a first binding domain specific for a first target; and the third protein includes a fourth heterodimerization sequence and a second binding domain specific for a second target. The first heterodimerization sequence binds the third heterodimerization sequence, and the second heterodimerization sequence binds the fourth heterodimerization sequence. In one embodiment, the first and second heterodimerization sequences are substantially identical and the third and fourth heterodimerization sequences are substantially identical. In another embodiment, the first and second heterodimerization sequences differ and the first heterodimerization sequence does not substantially bind the fourth heterodimerization sequence.

The first protein, for example, can include an Fc domain that includes at least two polypeptide chains. The Fc domain functions as the effector sequence. The two Fc polypeptide chains can be connected by a disulfide bond. The Fc domain can be glycosylated on at least an asparagine corresponding to asparagine 297 of CH2 (Kabat numbering). The effector sequence can be an Fc domain mutant, e.g., an asymmetric Fc domain and/or a modified specificity Fc domain.

Domains other than an Fc domain can be the effector sequence. The effector sequence can, for example, have one or more of the following properties: a) binding (e.g., specifically binding) to a surface of a cell, b) functionality in an extracellular environment, and c) detectability (i.e. other than being antigenic) (e.g., generates a signal). The effector sequence can include a human sequence or a modified human sequence.

In an embodiment, the effector sequence is glycosylated. In an embodiment, the effector sequence is a non-immunoglobulin effector sequence. The effector sequence, for example, can be an extracellular domain, or at least functional in the extracellular milieu.

Some effector sequences can bind to a cell surface, e.g., they recognize a cell surface receptor. Some effector sequences can elicit a cytotoxic effect. For example, the effector sequence can include a toxin. In another example, the effector sequences recruit a cytotoxic cell.

In an embodiment, the effector sequence includes a signal effector, e.g., a non-peptide label that is covalently attached to the second polypeptide. For example, the signal effector may be a contrast agent, e.g., an NMR contrast agent. For another example, the signal effector is a fluorescent protein.

The second and third proteins can each include an immunoglobulin variable domain. For example, they can each include Fab fragment or other antigen-binding domain. At least one of the immunoglobulin variable domains can include one or more synthetic CDRs, or naturally-occurring CDRs, e.g., a germline CDR and/or a somatic mutant thereof. In one embodiment, one or more of the CDRs is a human CDR, e.g., CDR3 is a human CDR. The immunoglobulin variable domains can include one or more human framework regions.

In one embodiment, the first and second targets are different proteins. In another embodiment, the first and second targets are different epitopes on the same protein. The first and/or second target can include a non-peptide compound that is recognized.

The first protein may be synthesized in a eukaryotic cell, e.g., a mammalian cell, e.g., a mammalian culture cell or in vivo. Further, the second and third proteins may be synthesized in vitro or in a bacterial cell. The second and third proteins can also, of course, be synthesized in a mammalian cell, and likewise the first protein may be synthesized in a bacterial cell or in vitro.

The invention also features nucleic acids that encode the afore-mentioned polypeptides, and kits that include the nucleic acids.

In another aspect, the invention features a method that includes: (a) evaluating a plurality of polypeptides to identify one or a set of polypeptides having a first property; and (b) for each member of the identified set or the one identified member, (i) attaching the member polypeptide to an effector polypeptide to form a complex; and (ii) determining a second property of the complex. The method can be used to screen the plurality of polypeptides, e.g., for a target binding property, and may be used in high throughput. The polypeptides of the plurality can vary. For example, the plurality can include at least 10², 10⁴, 10⁶, or 10⁸ species.

Typically, the attaching includes physically contacting the member polypeptide to the effector polypeptide. For example, the attaching can include binding a first interaction sequence linked to the member polypeptide to a second interaction sequence linked to the effector polypeptide. In another example, the attaching includes covalently coupling the member polypeptide to the effector polypeptide, e.g., by intein-mediated protein ligation. In still another example, the attaching includes binding the member polypeptide to the effector polypeptide using a hapten. For example, the effector polypeptide can be coupled to glutathione, and the member polypeptide attached to glutathione-S-transferase.

The first property, for example, can include a binding property. The second property can include one or more of a binding property, a cell-mediated property, and a cytotoxic activity. The first and second properties can be the same. In many implementations, the second property depends on the effector polypeptide.

In additional examples, the first property can include a catalytic activity, a biological activity (e.g., a physiological activity), a structural property and so forth.

The first and/or second property can be determined in vitro or in vivo. For example, the first property can be determined in vitro and the second property in vivo, and vice versa. In another embodiment, both properties are determined in vitro or in vivo. An in vivo determination can include monitoring a clinical outcome, in vivo imaging, monitoring a physiological property, and so forth.

In an embodiment, the plurality of polypeptides includes expression library members, e.g., display library members (e.g., cell or phage display library members).

The screening can include identifying first candidate polypeptides for the first property, and individually assaying each of the first candidate polypeptides to identify the one or the set of polypeptides. The screening can include expressing each polypeptide of the plurality in a prokaryotic cell. The effector sequence, for example, is expressed in a eukaryotic cell or an in vitro system. In some cases, the effector sequence is glycosylated.

The attaching can include expressing the member polypeptide in a prokaryotic cell.

The prokaryotic expression can include robotic manipulation for one or more of: preparing cultures of expressing cells; harvesting cultures of expressing cells; purifying polypeptides from the cells or media; and verifying polypeptide production by cells. The method can include individually assaying first candidate polypeptides using automated binding assays that generated values, and computer-based analysis of the generated values to identify the one or the set of polypeptides.

During the screening, each polypeptide of the plurality can be attached to a bacteriophage. In one embodiment, while determining the second property, each member polypeptide is not attached to a bacteriophage. In another embodiment, while determining the second property, each member polypeptide is attached to a bacteriophage.

The effector sequence can have one or more of the following properties: a) binds (e.g., specifically binds) to a surface of a cell, b) is functional in an extracellular environment, and c) is a detectable label (i.e. other than being antigenic) (e.g., generates a signal).

The effector polypeptide can include, e.g., a domain of an extracellular protein or an extracellular portion of a naturally-occurring protein. The effector polypeptide can be associated with one or more other polypeptide chains, e.g., which together form an effector sequence. For example, the effector sequence can include an immunoglobulin effector sequence (e.g., a domain that includes CH2) or a non-immunoglobulin effector sequence. In an embodiment, the effector sequence is glycosylated. For example, the effector polypeptide may be synthesized in a eukaryotic cell, e.g., a mammalian cell, e.g., a mammalian culture cell or in vivo.

In an embodiment, the effector sequence includes an Fc domain, e.g., CH2 and CH3 domains, e.g., IgG CH2 and IgG CH3 domains. The Fc domain can be glycosylated on at least an asparagine corresponding to asparagine 297 of CH2 (Kabat numbering). The effector sequence can be an Fc domain mutant, e.g., an asymmetric Fc domain and/or a modified specificity Fc domain.

The member polypeptide(s) and/or effector polypeptide can include a flexible region that spaces the interaction sequence from a domain of the member polypeptide or the effector sequence. In one embodiment, the flexible region includes an immunoglobulin hinge domain. The effector sequence can be N-terminal or C-terminal to the second interaction sequence.

In an embodiment, the effector sequence is a non-immunoglobulin effector sequence. The effector sequence, for example, can be an extracellular domain, or at least functional in the extracellular milieu.

Some effector sequences can bind to a cell surface, e.g., they recognize a cell surface receptor. Some effector sequences can elicit a cytotoxic effect. For example, the effector sequence can include a toxin.

In an embodiment, the effector sequence includes a signal effector, e.g., a non-peptide label that is covalently attached to the second polypeptide. For example, the signal effector may be a contrast agent, e.g., an NMR contrast agent. For another example, the signal effector is a fluorescent protein.

In one embodiment, the member polypeptide does not include an immunoglobulin domain. For example, the member polypeptide can include a cytokine, a peptide hormone, or a fragment thereof. In a related example, the member polypeptide includes a naturally occurring extracellular domain, e.g., a domain that includes a disulfide bond.

In another embodiment, the member polypeptide includes an immunoglobulin domain, e.g., an immunoglobulin variable domain. The member polypeptide can include an antigen-binding domain or fragment thereof. For example, the member polypeptide can include a VH and/or VL domain. Typically the member polypeptide is at least a component of the antigen-binding domain, e.g., in conjunction with another polypeptide. The antigen-binding domain can include the first immunoglobulin domain and a second immunoglobulin domain. The second immunoglobulin domain can be a component of the other polypeptide. The first and second immunoglobulin domains are generally variable domains. For example, the first immunoglobulin can be VH and the second immunoglobulin domain can be VL, or vice versa. In one embodiment, the member polypeptide includes both the VH and VL domain, e.g., a scFv. In another embodiment which includes the other polypeptide, the member polypeptide further includes a CH1 domain and the third polypeptide further includes a CL domain. The member and other polypeptide can be covalently linked by a disulfide bond. The first immunoglobulin variable domain can include one or more synthetic CDRs, or naturally-occurring CDRs, e.g., a germline CDR and/or a somatic mutant thereof. In one embodiment, one or more of the CDRs is a human CDR, e.g., CDR3 is a human CDR. The first immunoglobulin variable domain can include one or more human framework regions.

In another embodiment, the member polypeptide includes synthetic or includes a synthetic region. For example, the synthetic region can be about 6 to 30 amino acids, or longer. In one embodiment, the synthetic region includes a cysteine loop of about 4 to 15 amino acids. In still another embodiment, the member polypeptide includes a modified scaffold domain. Further, the member polypeptide can be a region of a naturally-occurring protein, e.g., a region of a mammalian ectodomain.

The first and second interaction sequences can be complementary heterodimerization sequences. For example, the first and second interaction sequences can be segments of single folded unit. In another example, the first and second interaction sequences are components of a coiled-coil. Such sequences can include a heptad repeat (typically at least 3, 4, or 5 repeats). They can be leucine zippers, e.g., the leucine zippers of fos and jun. They can have an amino acid sequence with fewer than 7, 4, 3, 2, or 1 substitutions relative to SEQ ID NOS: 1 to 8.

The member and effector polypeptides can each include a cysteine that forms a disulfide bond with the corresponding cysteine on the other polypeptide, e.g., when the first and second interaction sequences interact.

The member and/or effector polypeptide can further include a purification tag (the same or different tag).

In an embodiment, the member polypeptide includes a multimer of interaction sequences. For example, the multimer can include two, three, four, five, eight or more repeated units. One or more (e.g., at least two, three, up to and including all) of which is the first interaction sequence. Each of the first interaction sequences can be bound by a replicate of the second polypeptide, e.g., such that one or more replicates of the effector polypeptide are bound. Likewise, the effector polypeptide can include a multimer of interaction sequences, one or more of which is the second interaction sequence.

In another aspect, the invention features a method that includes: (a) a plurality of polypeptides to identify one or a set of polypeptides having a first property; and (b) for each member of the identified set or the one identified member, (i) attaching the member polypeptide to a target-binding sequence polypeptide to form a complex; and (ii) determining a second property of the complex. The method can be used to screen the plurality of polypeptides, e.g., for an effector property, and may be used in high throughput. The polypeptides of the plurality can vary. For example, the plurality can include at least 10², 10⁴, 10⁶, or 10⁸ species.

Typically, the attaching includes physically contacting the member polypeptide to the target-binding sequence polypeptide.. For example, the attaching can include binding a first interaction sequence linked to the member polypeptide to a second interaction sequence linked to the target-binding sequence polypeptide. In another example, the attaching includes covalently coupling the member polypeptide to the target-binding sequence polypeptide, e.g., by intein-mediated protein ligation. In still another example, the attaching includes binding the member polypeptide to the target-binding sequence polypeptide using a hapten. For example, the target-binding sequence polypeptide can be coupled to glutathione, and the member polypeptide attached to glutathione-S-transferase.

The first property, for example, can include a binding property, e.g., binding to an effector cell, e.g., a cell that expresses an Fc receptor. The second property can include one or more of a binding property, a cell-mediated property, and a cytotoxic activity. The first and second properties can be the same. In many implementations, the second property depends on the target-binding sequence polypeptide.

In additional examples, the first property can include a catalytic activity, a biological activity (e.g., a physiological activity), a structural property and so forth.

The first and/or second property can be determined in vitro or in vivo. For example, the first property can be determined in vitro and the second property in vivo, and vice versa. In another embodiment, both properties are determined in vitro or in vivo. An in vivo determination can include monitoring a clinical outcome, in vivo imaging, monitoring a physiological property, and so forth.

In an embodiment, the plurality of polypeptides includes expression library members, e.g., display library members (e.g., cell or phage display library members).

The screening can include identifying first candidate polypeptides for the first property, and individually assaying each of the first candidate polypeptides to identify the one or the set of polypeptides. The screening can include expressing each polypeptide of the plurality in a prokaryotic or eukaryotic cell. The targeting binding domain polypeptide, for example, is expressed in a eukaryotic cell or an in vitro system. In some cases, the member polypeptide is glycosylated.

The screening can include robotic manipulation for one or more of: preparing cultures of expressing cells; harvesting cultures of expressing cells; purifying polypeptides from the cells or media; and verifying polypeptide production by cells. The method can include individually assaying first candidate polypeptides using automated binding assays that generated values, and computer-based analysis of the generated values to identify the one or the set of polypeptides.

During the screening, each polypeptide of the plurality can be attached to a bacteriophage. In one embodiment, while determining the second property, each member polypeptide is not attached to a bacteriophage. In another embodiment, while determining the second property, each member polypeptide is attached to a bacteriophage.

The member polypeptide can be screened for a property that requires one or more of the following: a) binding (e.g., specifically binding) to a surface of a cell, b) functionality in an extracellular environment, and c) detectability (i.e. other than being antigenic) (e.g., generates a signal).

In an embodiment, the effector polypeptide includes a domain of an extracellular protein or an extracellular portion of a naturally-occurring protein. For example, the method is used to screen a cDNA library that includes extracellular domains. The member polypeptide can be associated with one or more other polypeptide chains, e.g., which together form a domain. In one embodiment, the member polypeptide lacks a functional immunoglobulin variable domain. In another embodiment, the member polypeptide includes a functional immunoglobulin variable domain.

In an embodiment, the member polypeptide includes an Fc domain, e.g., CH2 and CH3 domains, e.g., IgG CH2 and IgG CH3 domains. The method is used to screen for Fc domains with altered specificity or properties.

In an embodiment, the member polypeptide is a non-immunoglobulin effector sequence. The effector sequence, for example, can be an extracellular domain, or at least functional in the extracellular milieu.

Member polypeptide scan be identified that bind to a cell surface, e.g., they recognize a cell surface receptor and/or that elicit a cytotoxic effect.

In one embodiment, the target-binding sequence polypeptide does not include an immunoglobulin domain. For example, the target-binding sequence polypeptide can include a cytokine, a peptide hormone, or a fragment thereof. In a related example, the target-binding sequence polypeptide includes a naturally occurring extracellular domain, e.g., a domain that includes a disulfide bond.

In another embodiment, the target-binding sequence polypeptide includes an immunoglobulin domain, e.g., an immunoglobulin variable domain. The target-binding sequence polypeptide can include an antigen-binding domain or fragment thereof. For example, the target-binding sequence polypeptide can include a VH and/or VL domain. Typically the target-binding sequence polypeptide is at least a component of the antigen-binding domain, e.g., in conjunction with another polypeptide. The antigen-binding domain can include the first immunoglobulin domain and a second immunoglobulin domain. The second immunoglobulin domain can be a component of the other polypeptide. The first and second immunoglobulin domains are generally variable domains. For example, the first immunoglobulin can be VH and the second immunoglobulin domain can be VL, or vice versa. In one embodiment, the target-binding sequence polypeptide includes both the VH and VL domain, e.g., a scFv. In another embodiment which includes the other polypeptide, the target-binding sequence polypeptide further includes a CH1 domain and the third polypeptide further includes a CL domain. The member and other polypeptide can be covalently linked by a disulfide bond. The first immunoglobulin variable domain can include one or more synthetic CDRs, or naturally-occurring CDRs, e.g., a germline CDR and/or a somatic mutant thereof. In one embodiment, one or more of the CDRs is a human CDR, e.g., CDR3 is a human CDR. The first immunoglobulin variable domain can include one or more human framework regions.

In another embodiment, the target-binding sequence polypeptide includes a synthetic region. For example, the synthetic region can be about 6 to 30 amino acids, or longer. In one embodiment, the synthetic region includes a cysteine loop of about 4 to 15 amino acids. In still another embodiment, the target-binding sequence polypeptide includes a modified scaffold domain. Further, the target-binding sequence polypeptide can be a region of a naturally-occurring protein, e.g., a region of a mammalian ectodomain.

The first and second interaction sequences can be complementary heterodimerization sequences. For example, the first and second interaction sequences can be segments of single folded unit. In another example, the first and second interaction sequences are components of a coiled-coil. Such sequences can include a heptad repeat (typically at least 3, 4, or 5 repeats). They can be leucine zippers, e.g., the leucine zippers of fos and jun. They can have an amino acid sequence with fewer than 7, 4, 3, 2, or 1 substitutions relative to SEQ ID NOS: 1 to 8.

The member and target-binding sequence polypeptides can each include a cysteine that forms a disulfide bond with the corresponding cysteine on the other polypeptide, e.g., when the first and second interaction sequences interact.

The member and/or target-binding sequence polypeptides can further include a purification tag (the same or different tag).

The member polypeptide(s) and/or target binding polypeptide can include a flexible region that spaces the interaction sequence from a domain of the member polypeptide or the effector sequence. In one embodiment, the flexible region includes an immunoglobulin hinge domain. The target binding polypeptide can be N-terminal or C-terminal to the second interaction sequence.

In an embodiment, the target-binding sequence polypeptide includes a multimer of interaction sequences. For example, the multimer can include two, three, four, five, eight or more repeated units. One or more (e.g., at least two, three, up to and including all) of which is the first interaction sequence. Each of the first interaction sequences can be bound by a replicate of the second polypeptide, e.g., such that one or more replicates of the effector polypeptide are bound. Likewise, the effector polypeptide can include a multimer of interaction sequences, one or more of which is the second interaction sequence.

In another aspect, the invention features a method that includes: (a) providing (1) a first plurality of polypeptides, each polypeptide of the first plurality including a first polypeptide segment and a first interaction sequence, the first polypeptide segments varying among the first plurality, and (2) a second plurality of polypeptides, each polypeptide of the second plurality including a second polypeptide segment and a second interaction sequence that binds to the first interaction sequence, the second polypeptide segments varying among the second plurality; (b) contacting each polypeptide of the first plurality and each polypeptide of the second plurality to form complexes; and (c) evaluating each complex for an activity that depends on the respective first polypeptide segment and the second polypeptide segment. The method can be used, for example, as a library-against-library screen.

Generally, the evaluated activity is an activity that can function outside of the cell. For example, the activity can include binding to a cell surface, e.g., by one or both of the polypeptide segments, e.g., binding to a cell surface protein or insertion of a polypeptide into or through the cell surface.

The complex is typically a soluble, protein complex (of one or more polypeptide chains).

The contacting can include binding the first interaction sequence to the second interaction sequence.

In another aspect, the invention features a method that includes: providing (1) a plurality of first polypeptides, each first polypeptide including a first polypeptide segment and a first interaction sequence, the first polypeptide segments varying among the plurality, and (2) a second polypeptide including a second polypeptide segment and a second interaction sequence that binds to the first interaction sequence; contacting each polypeptide of the plurality to the second polypeptide to form a plurality of complexes; assaying each complex for an activity that depends on the respective first polypeptide segment and the second polypeptide segment, wherein the second polypeptide segment has one or more the following properties: a) binding (e.g., specifically binding) to a surface of a cell, b) functionality in an extracellular environment, and c) detectability (i.e. other than being antigenic) (e.g., generates a signal). The second polypeptide segment can include an effector sequence or a target-binding sequence.

At least one of the first polypeptide segments may also bind to a target molecule, e.g., an extracellular molecule. The first plurality can include at least 20, 50, 100, 200, 500, or 1000 entities.

In one embodiment, the first polypeptide segment includes a target-binding sequence. In a related embodiment, the second polypeptide segment includes an effector sequence.

The first polypeptide segment target-binding sequence can includes an immunoglobulin domain, e.g., an immunoglobulin variable domain. The target-binding sequence can include an antigen-binding domain. For example, the first polypeptide can include a VH and/or VL domain. Typically the first polypeptide is at least a component of the antigen-binding domain, e.g., in conjunction with a third polypeptide. The antigen-binding domain can include the first immunoglobulin domain and a second immunoglobulin domain. The second immunoglobulin domain can be a component of the third polypeptide. The first and second immunoglobulin domains are generally variable domains. For example, the first immunoglobulin can be VH and the second immunoglobulin domain can be VL, or vice versa. In one embodiment, the first polypeptide includes both the VH and VL domain, e.g., a scFv. In another embodiment which includes the third polypeptide, the first polypeptide further includes a CH1 domain and the third polypeptide further includes a CL domain. The first and third polypeptide can be covalently linked by a disulfide bond. The first immunoglobulin variable domain can include one or more synthetic CDRs, or naturally-occurring CDRs, e.g., a germline CDR and/or a somatic mutant thereof. In one embodiment, one or more of the CDRs is a human CDR, e.g., CDR3 is a human CDR. The first immunoglobulin variable domain can include one or more human framework regions.

In another embodiment, the target-binding sequence is synthetic or includes a synthetic region. For example, the synthetic region can be about 6 to 30 amino acids, or longer. In one embodiment, the synthetic region includes a cysteine loop of about 4 to 15 amino acids. In still another embodiment, the target-binding sequence includes a modified scaffold domain. Further, the target-binding sequence can be a region of a naturally-occurring protein, e.g., a region of a mammalian ectodomain.

In one embodiment, the target-binding sequence and/or the effector sequence is not antigenic or immuno-reactive in humans. The target-binding sequence and/or effector sequence can include a human sequence or a modified human sequence.

The second polypeptide segment effector sequence can include, e.g., a domain of an extracellular protein or an extracellular portion of a naturally-occurring protein. The effector sequence can include one or more polypeptide chains, of which one (or more) is a component of the second polypeptide. For example, the effector sequence can include an immunoglobulin effector sequence (e.g., a domain that includes CH2) or a non-immunoglobulin effector sequence.

In an embodiment, the effector sequence is glycosylated. For example, the second polypeptide may be synthesized in a eukaryotic cell, e.g., a mammalian cell, e.g., a mammalian culture cell or in vivo. Further, the first polypeptide may be synthesized in vitro or in a bacterial cell. The first polypeptide can also, of course, be synthesized in a mammalian cell, and likewise the second polypeptide may be synthesized in a bacterial cell or in vitro.

In an embodiment, the effector sequence includes an Fc domain, e.g., CH2 and CH3 domains, e.g., IgG CH2 and IgG CH3 domains. The Fc domain can be glycosylated on at least an asparagine corresponding to asparagine 297 of CH2 (Kabat numbering). The effector sequence can be an Fc domain mutant, e.g., an asymmetric Fc domain and/or a modified specificity Fc domain.

In an embodiment, the effector sequence is a non-immunoglobulin effector sequence. The effector sequence, for example, can be an extracellular domain, or at least functional in the extracellular milieu.

Some effector sequences can bind to a cell surface, e.g., they recognize a cell surface receptor. Some effector sequences can elicit a cytotoxic effect. For example, the effector sequence can include a toxin.

In an embodiment, the effector sequence includes a signal effector, e.g., a non-peptide label that is covalently attached to the second polypeptide. For example, the signal effector may be a contrast agent, e.g., an NMR contrast agent. For another example, the signal effector is a fluorescent protein.

The first and second interaction sequences can be complementary heterodimerization sequences. For example, the first and second interaction sequences can be segments of single folded unit. In another example, the first and second interaction sequences are components of a coiled-coil. Such sequences can include a heptad repeat (typically at least 3, 4, or 5 repeats). They can be leucine zippers, e.g., the leucine zippers of fos and jun. They can have an amino acid sequence with fewer than 7, 4, 3, 2, or 1 substitutions relative to SEQ ID NOS: 1 to 8.

The first and second polypeptides can each include a cysteine that forms a disulfide bond with the corresponding cysteine on the other polypeptide, e.g., when the first and second interaction sequences interact.

The first and/or second polypeptide can further include a purification tag (the same or different tag). The protein can also include a non-peptide conjugate.

In an embodiment, the first polypeptide includes a multimer of interaction sequences. For example, the multimer can include two, three, four, five, eight or more repeated units. One or more (e.g., at least two, three, up to and including all) of which is the first interaction sequence. Each of the first interaction sequences can be bound by a replicate of the second polypeptide, e.g., such that one or more replicates of the second polypeptide are components of the protein. Likewise, the second polypeptide can include a multimer of interaction sequences, one or more of which is the second interaction sequence.

The first and/or second polypeptide can include a flexible region that spaces the interaction sequence from the target-binding sequence or the effector sequence. In one embodiment, the flexible region includes an immunoglobulin hinge domain. The effector sequence can be N-terminal or C-terminal to the second interaction sequence.

In yet another aspect, the invention features a method that includes: providing a cell that includes a nucleic acid that encodes a first polypeptide that includes a target-binding sequence (e.g., an immunoglobulin variable domain) and a first interaction sequence; culturing the cell under conditions such that the first polypeptide is expressed; optionally, isolating the first polypeptide from first cell; and contacting the polypeptide to a second polypeptide (or effector polypeptide) that includes a second interaction sequence and an effector sequence to form a complex.

The method can include, prior to the contacting, expressing the second polypeptide in the first cell or in a second cell. The second cell can be a prokaryotic or eukaryotic cell.

The first cell can be a prokaryotic or eukaryotic cell. The first cell can secrete the first polypeptide. In one implementation, the first and second cell are co-cultured. The second cell can secrete the second polypeptide.

The target-binding sequence can include an immunoglobulin domain, e.g., an immunoglobulin variable domain. The target-binding sequence can include an antigen-binding domain. For example, the first polypeptide can include a VH and/or VL domain. Typically the first polypeptide is at least a component of the antigen-binding domain, e.g., in conjunction with a third polypeptide. The antigen-binding domain can include the first immunoglobulin domain and a second immunoglobulin domain. The second immunoglobulin domain can be a component of the third polypeptide. The first and second immunoglobulin domains are generally variable domains. For example, the first immunoglobulin can be VH and the second immunoglobulin domain can be VL, or vice versa. In one embodiment, the first polypeptide includes both the VH and VL domain, e.g., a scFv. In another embodiment which includes the third polypeptide, the first polypeptide further includes a CH1 domain and the third polypeptide further includes a CL domain. The first and third polypeptide can be covalently linked by a disulfide bond. The first immunoglobulin variable domain can include one or more synthetic CDRs, or naturally-occurring CDRs, e.g., a germline CDR and/or a somatic mutant thereof In one embodiment, one or more of the CDRs is a human CDR, e.g., CDR3 is a human CDR. The first immunoglobulin variable domain can include one or more human framework regions.

In another embodiment, the target-binding sequence is synthetic or includes a synthetic region. For example, the synthetic region can be about 6 to 30 amino acids, or longer. In one embodiment, the synthetic region includes a cysteine loop of about 4 to 15 amino acids. In still another embodiment, the target-binding sequence includes a modified scaffold domain. Further, the target-binding sequence can be a region of a naturally-occurring protein, e.g., a region of a mammalian ectodomain.

In one embodiment, the target-binding sequence and/or the effector sequence is not antigenic or immuno-reactive in humans. The target-binding sequence and/or effector sequence can include a human sequence or a modified human sequence.

The effector sequence can include, e.g., a domain of an extracellular protein or an extracellular portion of a naturally-occurring protein. The effector sequence can include one or more polypeptide chains, of which one (or more) is a component of the second polypeptide. For example, the effector sequence can include an immunoglobulin effector sequence (e.g., a domain that includes CH2) or a non-immunoglobulin effector sequence.

In an embodiment, the effector sequence is glycosylated. For example, the second polypeptide may be synthesized in a eukaryotic cell, e.g., a mammalian cell, e.g., a mammalian culture cell or in vivo. Further, the first polypeptide may be synthesized in vitro or in a bacterial cell. The first polypeptide can also, of course, be synthesized in a mammalian cell, and likewise the second polypeptide may be synthesized in a bacterial cell or in vitro.

In an embodiment, the effector sequence includes an Fc domain, e.g., CH2 and CH3 domains, e.g., IgG CH2 and IgG CH3 domains. The Fc domain can be glycosylated on at least an asparagine corresponding to asparagine 297 of CH2 (Kabat numbering). The effector sequence can be an Fc domain mutant, e.g., an asymmetric Fc domain and/or a modified specificity Fc domain.

The first and/or second polypeptide can include a flexible region that spaces the interaction sequence from the target-binding sequence or the effector sequence. In one embodiment, the flexible region includes an immunoglobulin hinge domain. The effector sequence can be N-terminal or C-terminal to the second interaction sequence.

In an embodiment, the effector sequence is a non-immunoglobulin effector sequence. The effector sequence, for example, can be an extracellular domain, or at least functional in the extracellular milieu.

Some effector sequences can bind to a cell surface, e.g., they recognize a cell surface receptor. Some effector sequences can elicit a cytotoxic effect. For example, the effector sequence can include a toxin.

In an embodiment, the effector sequence includes a signal effector, e.g., a non-peptide label that is covalently attached to the second polypeptide. For example, the signal effector may be a contrast agent, e.g., an NMR contrast agent. For another example, the signal effector is a fluorescent protein.

The first and second interaction sequences can be complementary heterodimerization sequences. For example, the first and second interaction sequences can be segments of single folded unit. In another example, the first and second interaction sequences are components of a coiled-coil. Such sequences can include a heptad repeat (typically at least 3, 4, or 5 repeats). They can be leucine zippers, e.g., the leucine zippers of fos and jun. They can have an amino acid sequence with fewer than 7, 4, 3, 2, or 1 substitutions relative to SEQ ID NOS: 1 to 8.

The first and second polypeptides can each include a cysteine that forms a disulfide bond with the corresponding cysteine on the other polypeptide, e.g., when the first and second interaction sequences interact.

The first and/or second polypeptide can further include a purification tag (the same or different tag). The protein can also include a non-peptide conjugate.

In an embodiment, the first polypeptide includes a multimer of interaction sequences. For example, the multimer can include two, three, four, five, eight or more repeated units. One or more (e.g., at least two, three, up to and including all) of which is the first interaction sequence. Each of the first interaction sequences can be bound by a replicate of the second polypeptide, e.g., such that one or more replicates of the second polypeptide are components of the protein. Likewise, the second polypeptide can include a multimer of interaction sequences, one or more of which is the second interaction sequence.

In another aspect, the invention features a method that includes: providing a vector nucleic acid that includes: (1) an insert site; and (2) a segment encoding a interaction sequence, and optionally (3) a segment encoding a signal (e.g., secretory) sequence; inserting a nucleic acid encoding a target-binding sequence into the insert site, such that the nucleic acid encoding the target-binding sequence and the segment encoding the interaction sequence are in frame; synthesizing a first polypeptide encoded by the vector nucleic acid that includes the target-binding sequence and the first interaction sequence; contacting the first polypeptide to a second polypeptide that includes an effector sequence and a second interaction sequence to form a complex.

The contacting can be in vitro or outside a cell (e.g., in media or a purified environment). The method can further include, after the contacting, assaying the complex for a functional activity. The method can further include assaying the first polypeptide for a binding activity, e.g., prior to the combining. The binding assay can be before or after the inserting.

In an embodiment, the synthesizing is in vitro. In another embodiment, the synthesizing is in vivo, e.g., in a bacterial, yeast, or mammalian cell.

In an embodiment, the first polypeptide is not glycosylated.

In an embodiment, the functional activity is a cytotoxic activity, e.g., complement mediated cytotoxicity or antibody dependent cell-mediated cytotoxicity.

The vector nucleic acid can further include a sequence encoding a purification tag that is bindable to a moiety. The method can further include binding the first polypeptide to a moiety attached to a solid support. For example, the second interaction sequence can be attached to the solid support. The contacting can include eluting the first polypeptide from the solid support using the effector polypeptide.

The target-binding sequence can includes an immunoglobulin domain, e.g., an immunoglobulin variable domain. The target-binding sequence can include an antigen-binding domain. For example, the first polypeptide can include a VH and/or VL domain. Typically the first polypeptide is at least a component of the antigen-binding domain, e.g., in conjunction with a third polypeptide. The antigen-binding domain can include the first immunoglobulin domain and a second immunoglobulin domain. The second immunoglobulin domain can be a component of the third polypeptide. The first and second immunoglobulin domains are generally variable domains. For example, the first immunoglobulin can be VH and the second immunoglobulin domain can be VL, or vice versa. In one embodiment, the first polypeptide includes both the VH and VL domain, e.g., a scFv. In another embodiment which includes the third polypeptide, the first polypeptide further includes a CH1 domain and the third polypeptide further includes a CL domain. The first and third polypeptide can be covalently linked by a disulfide bond. The first immunoglobulin variable domain can include one or more synthetic CDRs, or naturally-occurring CDRs, e.g., a germline CDR and/or a somatic mutant thereof. In one embodiment, one or more of the CDRs is a human CDR, e.g., CDR3 is a human CDR. The first immunoglobulin variable domain can include one or more human framework regions.

In another embodiment, the target-binding sequence is synthetic or includes a synthetic region. For example, the synthetic region can be about 6 to 30 amino acids, or longer. In one embodiment, the synthetic region includes a cysteine loop of about 4 to 15 amino acids. In still another embodiment, the target-binding sequence includes a modified scaffold domain. Further, the target-binding sequence can be a region of a naturally-occurring protein, e.g., a region of a mammalian ectodomain.

In one embodiment, the target-binding sequence and/or the effector sequence is not antigenic or immuno-reactive in humans. The target-binding sequence and/or effector sequence can include a human sequence or a modified human sequence.

The effector sequence can include, e.g., a domain of an extracellular protein or an extracellular portion of a naturally-occurring protein. The effector sequence can include one or more polypeptide chains, of which one (or more) is a component of the second polypeptide. For example, the effector sequence can include an immunoglobulin effector sequence (e.g., a domain that includes CH2) or a non-immunoglobulin effector sequence.

In an embodiment, the effector sequence is glycosylated. For example, the second polypeptide may be synthesized in a eukaryotic cell, e.g., a mammalian cell, e.g., a mammalian culture cell or in vivo. Further, the first polypeptide may be synthesized in vitro or in a bacterial cell. The first polypeptide can also, of course, be synthesized in a mammalian cell, and likewise the second polypeptide may be synthesized in a bacterial cell or in vitro.

In an embodiment, the effector sequence includes an Fc domain, e.g., CH2 and CH3 domains, e.g., IgG CH2 and IgG CH3 domains. The Fc domain can be glycosylated on at least an asparagine corresponding to asparagine 297 of CH2 (Kabat numbering). The effector sequence can be an Fc domain mutant, e.g., an asymmetric Fc domain and/or a modified specificity Fc domain.

In an embodiment, the effector sequence is a non-immunoglobulin effector sequence. The effector sequence, for example, can be an extracellular domain, or at least functional in the extracellular milieu.

Some effector sequences can bind to a cell surface, e.g., they recognize a cell surface receptor. Some effector sequences can elicit a cytotoxic effect. For example, the effector sequence can include a toxin.

In an embodiment, the effector sequence includes a signal effector, e.g., a non-peptide label that is covalently attached to the second polypeptide. For example, the signal effector may be a contrast agent, e.g., an NMR contrast agent. For another example, the signal effector is a fluorescent protein.

The first and second interaction sequences can be complementary heterodimerization sequences. For example, the first and second interaction sequences can be segments of single folded unit. In another example, the first and second interaction sequences are components of a coiled-coil. Such sequences can include a heptad repeat (typically at least 3, 4, or 5 repeats). They can be leucine zippers, e.g., the leucine zippers of fos and jun. They can have an amino acid sequence with fewer than 7, 4, 3, 2, or 1 substitutions relative to SEQ ID NOS: 1 to 8.

The first and second polypeptides can each include a cysteine that forms a disulfide bond with the corresponding cysteine on the other polypeptide, e.g., when the first and second interaction sequences interact.

The first and/or second polypeptide can further include a purification tag (the same or different tag). The protein can also include a non-peptide conjugate.

In an embodiment, the first polypeptide includes a multimer of interaction sequences. For example, the multimer can include two, three, four, five, eight or more repeated units. One or more (e.g., at least two, three, up to and including all) of which is the first interaction sequence. Each of the first interaction sequences can be bound by a replicate of the second polypeptide, e.g., such that one or more replicates of the second polypeptide are components of the protein. Likewise, the second polypeptide can include a multimer of interaction sequences, one or more of which is the second interaction sequence.

The first and/or second polypeptide can include a flexible region that spaces the interaction sequence from the target-binding sequence or the effector sequence. In one embodiment, the flexible region includes an immunoglobulin hinge domain. The effector sequence can be N-terminal or C-terminal to the second interaction sequence.

In another aspect, the invention features a method that includes: identifying a member of a display library, wherein the member includes a nucleic acid that includes (i) a segment encoding a polypeptide that includes a first immunoglobulin domain and a first interaction sequence, (ii) a suppressible stop codon, and (iii) a display library element; introducing the nucleic acid of the identified member into a bacterial host cell; culturing the bacterial host cell under conditions such that the nucleic acid is expressed and the cell synthesizes the polypeptide in a form that is not attached to the display library element; and binding the synthesized polypeptide to a purified effector polypeptide that includes an effector sequence and a second interaction sequence that binds the first interaction sequence.

The effector sequence can, for example, be an effector sequence described herein. The effector polypeptide can be glycosylated. For example, the effector polypeptide is expressed by a mammalian host cell. The interaction sequences can, for example, be any interaction sequences described herein.

In an embodiment, the bacterial host cell lacks a suppressor tRNA gene.

In an embodiment, the display library is a phage display library. The display library element can encode one or more amino acids that attach the encoded polypeptide (directly or indirectly) to the display library nucleic acid.

In yet another aspect, the invention features a cell that includes (1) a heterologous surface-attached protein having a first interaction sequence, and (2) a second protein that includes a second interaction sequence and is bound to the first interaction sequence. The second protein also includes a subject sequence, in addition to the second interaction sequence. The subject sequence can be at least a part of a target-binding sequence, e.g., an antigen-binding domain. The surface-attached protein is heterologous with respect to the cell, but may be naturally occurring protein. Typically the surface-attached protein is not naturally-occurring. The second interaction sequence does not include an immunoglobulin variable domain.

The cell can be a prokaryotic or eukaryotic cell, e.g., a yeast or mammalian cell. In an embodiment, the cell is an immune cell, e.g., a CTL, killer cell, NK cell, macrophage, monocytes, eosinophils, neutrophil, polymorphonuclear cell, granulocyte, mast cell, or basophil. The first and second interaction sequences can be complementary heterodimerization sequences. For example, the first and second interaction sequences can be segments of single folded unit. In another example, the first and second interaction sequences are components of a coiled-coil. Such sequences can include a heptad repeat (typically at least 3, 4, or 5 repeats). They can be leucine zippers, e.g., the leucine zippers of fos and jun. They can have an amino acid sequence with fewer than 7, 4, 3, 2, or 1 substitutions relative to SEQ ID NOS: 1 to 8.

In one embodiment, the interaction sequences are intracellular interaction sequences (e.g., fos and jun). In another embodiment, the interaction sequences are extracellular interaction sequences (e.g., Notch and Delta ectodomains).

The surface-attached protein and the second protein can each include a cysteine that forms a disulfide bond with the corresponding cysteine on the other polypeptide, e.g., when the first and second interaction sequences interact.

In one embodiment, the surface-attached protein includes at least one transmembrane domain. In another embodiment, the surface-attached protein is covalently linked to a plasma membrane lipid, e.g., a phosphoinositol linkage.

In an embodiment, the second protein includes a modified scaffold domain, a cysteine loop peptide, a linear peptide sequence, and/or a synthetic polypeptide sequence. The modified scaffold domain can be, e.g., an antibody variable domain. The immunoglobulin variable domain can include one or more synthetic CDRs, or naturally-occurring CDRs, e.g., a germline CDR and/or a somatic mutant thereof. In one embodiment, one or more of the CDRs is a human CDR, e.g., CDR3 is a human CDR. The first immunoglobulin variable domain can include one or more human framework regions.

In a related aspect, the invention features a cell that includes (1) a surface-attached protein having a first interaction sequence, and (2) an artificial second protein that includes a second interaction sequence, specifically bound to the first interaction sequence, and a subject sequence, independent of the second interaction sequence. The surface-attached protein can be a heterologous or endogenous protein. The second interaction sequence does not include an antibody variable domain.

The subject sequence can be at least a part of a target-binding sequence, e.g., an antigen-binding domain.

The cell can be a prokaryotic or eukaryotic cell, e.g., a yeast or mammalian cell. In an embodiment, the cell is an immune cell, e.g., a CTL, killer cell, NK cell, macrophage, monocytes, eosinophils, neutrophil, polymorphonuclear cell, granulocyte, mast cell, or basophil. The first and second interaction sequences can be complementary heterodimerization sequences. For example, the first and second interaction sequences can be segments of single folded unit. In another example, the first and second interaction sequences are components of a coiled-coil. Such sequences can include a heptad repeat (typically at least 3, 4, or 5 repeats). They can be leucine zippers, e.g., the leucine zippers of fos and jun. They can have an amino acid sequence with fewer than 7, 4, 3, 2, or 1 substitutions relative to SEQ ID NOS: 1 to 8.

In one embodiment, the interaction sequences are intracellular interaction sequences (e.g., fos and jun). In another embodiment, the interaction sequences are extracellular interaction sequences (e.g., Notch and Delta ectodomains).

The surface-attached protein and the second protein can each include a cysteine that forms a disulfide bond with the corresponding cysteine on the other polypeptide, e.g., when the first and second interaction sequences interact.

In one embodiment, the surface-attached protein includes at least one transmembrane domain. In another embodiment, the surface-attached protein is covalently linked to a plasma membrane lipid, e.g., a phosphoinositol linkage.

In an embodiment, the second protein includes a modified scaffold domain, a cysteine loop peptide, a linear peptide sequence, and/or a synthetic polypeptide sequence. The modified scaffold domain can be, e.g., an antibody variable domain. The immunoglobulin variable domain can include one or more synthetic CDRs, or naturally-occurring CDRs, e.g., a germline CDR and/or a somatic mutant thereof. In one embodiment, one or more of the CDRs is a human CDR, e.g., CDR3 is a human CDR. The first immunoglobulin variable domain can include one or more human framework regions.

In another aspect, the invention features a method that includes providing a cell that includes a heterologous surface-attached protein having a first interaction sequence, and contacting the cell with a second protein that includes a second interaction sequence and is bound to the first interaction sequence. The method can further include covalently linking the surface-attached protein and the second protein. The method can further include determining a cellular activity that depends on interaction of a domain of the second protein with a target, e.g., a target cell. The cellular activity can be cytotoxicity. The cellular activity can be determined in vivo. Further the second protein can be identified in an expression library, e.g., a display library prior to the contacting. In some implementations, no recloning or reformatting of the library member is required to attach the second protein to the cell.

The invention also features nucleic acids that encode the afore-mentioned polypeptides, host cells that include one or more of the nucleic acids, nucleic acid vectors, and kits that include the nucleic acids and/or nucleic acid vectors. Nucleic acid vectors can include a receiving site (e.g., a restriction enzyme polylinker or recombination sites) for inserting a nucleic acid sequence encoding at least a part of a target-binding or effector sequence.

One exemplary kit includes: (1) a first nucleic acid that includes a sequence that encodes a first polypeptide that includes a first immunoglobulin domain and a first interaction sequence, wherein the first interaction sequence specifically recognizes a second interaction sequence; and (2) one or more of: (i) a second polypeptide that includes the second interaction sequence and an effector sequence, or (ii) a second nucleic acid that includes a sequence that encodes the second polypeptide. The effector sequence does not include a functional immunoglobulin variable domain. Features of the encoded polypeptides are described above and herein. For example, the second polypeptide can include CH2 and CH3 domains and/or can be glycosylated. The second nucleic acid can be provided in a host cell, e.g., a bacterial or mammalian host cell. In an embodiment, the first and second nucleic acid are co-linear.

Another exemplary kit includes (1) a first nucleic acid vector that includes a site for receiving a sequence that encodes a first polypeptide and a sequence encoding a first interaction sequence, wherein insertion of the sequence encoding the first polypeptide into the vector can result in a translational fusion of the first polypeptide and the first interaction sequence; and

(2) one or more of: (i) a second polypeptide that includes an effector sequence and a second interaction sequence that is bound by the first interaction sequence, and (ii) a second nucleic acid that includes a sequence that encodes the second polypeptide.

In another example, the invention features a nucleic acid that includes a first and second segment. The first segment includes a sequence that encodes a first polypeptide that includes a first immunoglobulin domain and a first interaction sequence. The second segment includes a sequence that encodes a second polypeptide that includes a second interaction sequence and an effector sequence. The first interaction sequence interacts with (e.g., binds) the second interaction sequence. The first and second segment can be transcribed by the same promoter.

As used herein, a “protein” refers to a biological polymer that includes at least three amino acids in one or more polypeptide chains. In the case of two or more chains, the chains may be covalently or non-covalently associated. A “polypeptide” refers to a chain of at least three amino acids. A “peptide” refers to a chain of between three and thirty amino acids.

As used herein, a “domain” of a protein refers to a region with a particular property. A domain does not necessarily have an independently folded structure, although it can, i.e., it is an “independently folded domain.”

As used herein, an “interaction sequence” refers to a region of an amino acid sequence that can bind to another protein. The binding can be specific and of high affinity. Examples of interaction sequence include heterodimerization sequences and other hetero-oligomerization sequences (e.g., sequences that form trimers, tetramers, and so forth). An interaction sequence can, for example, form a unitary functional, folded unit. However, in some implementations, the interaction sequence can be distributed among discontinuous binding surfaces or discontinuous folded units of different polypeptides. Also included are interaction sequences (e.g., as described in U.S. Pat. No. 6,294,353) formed by separation of segments from a single folded unit.

As used herein, a “heterodimerization sequence” refers to a polypeptide sequence that can bind to another polypeptide sequence (i.e., its partner). The partner sequence is less than 98% identical (or less than 95%, 90%, 85%, 80%, or 70%) to the heterodimerization sequence. The first and second heterodimerization sequence can bind to each other, e.g., with a equilibrium dissociation constant of less than about 10⁻⁷ M, e.g., less than about 10⁻⁸, 10⁻⁹, or 10⁻¹⁰. In one embodiment, the first heterodimerization sequence can form a homodimer, but has a higher stability if it forms a heterodimers with the partner sequence. In a related embodiment, the population of dimers (both homodimers and heterodimers) is about 50% to 100%, 70% to 90%, or at least about 80% or 90% heterodimer.

The “effector sequence” can include an “effector domain” which any functional domain that can produce a signal or effect, or a functional segment thereof (e.g., a CH2 domain is an effector sequence). Non-limiting examples of effector domains include an immunological effector domain, a labelling domain, an enzymatic domain, or a non-immunoglobulin cell binding domain. One exemplary class of effector domains includes effector domains that are functional in the extracellular environment. Such domains differ, for example, from a transcriptional activation domain which functions within the nucleus of a eukaryotic cell.

An exemplary immunological effector domain includes the Fc domain. The Fc domain binds to host tissues or factors, including various cells of the immune system (e.g., effector cells) and the first component (C1q) of the classical complement system. The effector domain can include an Fc domain, e.g., CH2, CH3, CH4, CH2—CH3, and CH2—CH3—CH4. The effector domain can, in some implementations, include the hinge region, i.e., the region between CH1 and CH2.

The Fc can be a Fc dimer. The Fc domain can also be of any isotype (e.g., IgM, IgG1, IgG2, IgG3, or IgG4). In one embodiment, the Fc effector domain is glycosylated, e.g., at the asparagine corresponding to asparagine 297 of IgG (Kabat numbering). Preferably, the Fc domain can bind C1q, e.g., if aggregated, and can bind an Fc receptor, e.g., FCγR1, FCγRIIA, FCγRIIB, FCγRIIIA, or FCγRIIIB. In a related embodiment, when aggregated, the effector domain elicits a response, e.g., a cytotoxic response, from leukocytes, e.g., NK cells.

Other effector domains include domains that can produce signals, e.g., green fluorescent protein and derivatives thereof, luciferase, alkaline phosphatase, and horseradish peroxidase. Still other effector domains include a cytotoxin or cytotoxin component, e.g., a chain of diphtheria toxin, ricin, or cholera toxin. Many such effector domains can bind to a cell surface.

As used herein, “specific binding” refers to the property of a protein, e.g., a target or antigen-binding protein or domain: (1) to bind to a target with an affinity of at least 1×10⁷ M⁻¹, and (2) to preferentially bind to the target with an affinity that is at least two-fold greater than its affinity for binding to a non-specific target (e.g., BSA or casein)

As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

The antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (C1q) of the classical complement system. The term “antibody” includes intact immunoglobulins of types IgA, IgG, IgE, IgD, IgM (as well as subtypes thereof), wherein the light chains of the immunoglobulin may be of types kappa or lambda.

As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd or 214 amino acids) are encoded by a variable region gene at the N-terminus (about 110 amino acids) and a kappa or lambda constant region gene at the C-terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids).

An “immunoglobulin domain” refers to a domain from the variable or constant domain of immunoglobulin molecules. The term “immunoglobulin superfamily domain” is distinguished from “immunoglobulin domain.” An “immunoglobulin superfamily domain” refers to a domain that has a three-dimensional structure related to an immunoglobulin domain, but is from a non-immunoglobulin molecule. Immunoglobulin domains and immunoglobulin superfamily domains typically contains two β-sheets formed of about seven β-strands, and a conserved disulphide bond (see, e.g., Williams and Barclay 1988 Ann. Rev Immunol. 6:381-405). Proteins that include immunoglobulin superfamily domains include CD4, platelet derived growth factor receptor (PDGFR), and intercellular adhesion molecule (ICAM). Immunoglobulin superfamily domains from these proteins, for example, are consider non-immunoglobulin target-binding domains if they function to bind a specific target.

As used herein, “isotype” refers to the antibody class (e.g., IgM or IgG1) that is encoded by heavy chain constant region genes.

The term “antigen-binding fragment” of an antibody (or “antigen-binding domain”), as used herein, refers to one or more fragments of a full-length antibody that retain the ability to specifically bind to a target (e.g., an antigen such a polypeptide or a hapten). Examples of binding fragments encompassed within the term “antigen-binding fragment” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies.

A so-called “split antibody” refers to an antibody in which an effector domain and the antigen-binding domain are components of separate polypeptide chains.

An “effector cell” is an immune cell which is involved in the effector phase of an immune response. Exemplary immune cells include a cell of a myeloid or lymphoid origin, e.g., lymphocytes (e.g., B cells and T cells including cytolytic T cells (CTLs)), killer cells, natural killer cells, macrophages, monocytes, eosinophils, neutrophils, polymorphonuclear cells, granulocytes, mast cells, and basophils. Effector cells express specific Fc receptors and mediate specific immune functions. Some effector cells can induce antibody-dependent cellular toxicity (ADCC), e.g., a neutrophil capable of inducing ADCC. Monocytes, macrophages, neutrophils, eosinophils, and lymphocytes which express FcαR are involved in specific killing of target cells and presenting antigens to other components of the immune system, or binding to cells that present antigens. In other embodiments, an effector cell can phagocytose a target antigen, target cell, or microorganism. The expression of a particular FcR on an effector cell can be regulated by humoral factors such as cytokines. For example, expression of FcγRI has been found to be up-regulated by interferon gamma (IFN-γ). This enhanced expression increases the cytotoxic activity of FcγRI-bearing cells against targets.

As used herein, a “naturally-occurring” nucleic acid molecule refers to an RNA or DNA molecule having a nucleotide sequence that occurs in nature. For example a naturally occurring nucleic acid molecule can encode a natural protein. The term “artificial” is synonymous with “non-naturally occurring” with respect to available sequence information at the time of inquiry.

A “heterologous” sequence refers to a sequence which is introduced into a cell or into the context of a nucleic acid by artifice. A heterologous sequence may be a copy of an endogenous gene, but, for example, inserted into an exogenous plasmid or into a chromosomal site at a position other than its endogenous position.

As used herein, a “transgenic animal” is a non-human animal, such as a mammal (e.g., a rodent such as a rat or mouse) in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous gene has been altered by, e.g., by homologous recombination between the endogenous gene and an exogenous DNA molecule introduced into a cell of the animal, e.g., an embryonic cell of the animal, prior to development of the animal. The exogenous DNA, for example, can include a sequence that encodes one or more of an interaction sequence, an effector domain and a target-binding domain.

An “isolated” or “purified” polypeptide or protein is substantially free of cellular material or other contaminating proteins from the cell or tissue source from which the protein is derived, or substantially free from chemical precursors or other chemicals when chemically synthesized. “Substantially free” means that a preparation of a given protein is at least 10% pure.

All citations, including citations to publications, patents, and patent applications, are incorporated herein by reference in their entirety. The details of one or more exemplary embodiments of the invention are set forth in the accompanying drawings and the description below. Other features, objects, and advantages of the invention will be apparent from the examples of the description and drawings, and from the claims.

### BRIEF DESCRIPTION OF DRAWINGS

FIGS. 1, 2, 3, 4, 5A, 5B, 6, 7A, 7B, 8 are schematics of exemplary proteins in which target-binding and effector functions are provided by separate polypeptide chains and joined by a non-covalent interaction.

### DETAILED DESCRIPTION

Natural full-length antibodies, such as IgG, include an antigen-binding domain and an effector domain. A part of the antigen-binding domain and the effector domain are components of a single polypeptide chain—the heavy chain. This structure enables an organism's immune system to identify and attack non-self antigens. The antigen-binding domain identifies such antigens, whereas the effector domain interacts with factors such as complement and NK cells to mediate the attack.

The invention provides, in part, compositions and methods to generate functional target-binding proteins from at least two separate polypeptide chains. In one embodiment, the two separate chains are reconstituted by a non-covalent binding interaction mediated by an interaction sequence such as a heterodimerization sequence, e.g., c-fos and c-jun. In another embodiment, the separate chains are covalently joined.

These methods facilitate the translation of the antigen-binding domain and the effector domain from separate transcripts. This flexibility enables a variety of applications. For example, the antigen-binding domain and the effector domain can be purified from separate recombinant host cells. In another example, the antigen-binding domain and the effector domain are expressed from separate nucleic acids, e.g., different transgenes or plasmids that are either in the same cell or different cells. In still another example, one or both of the antigen-binding domain and the effector domain are expressed from separate DNA molecules in an in vitro transcription-translation extract.

In one embodiment, the antigen-binding domain is first screened for a first property, e.g., for a binding property, in the absence of the effector domain. Subsequently, the effector domain is attached, and the functionality of the complex is assayed, e.g., for a second property, e.g., a property dependent on the effector domain. This strategy is appropriate for screening numerous bacterially produced antigen-binding domains for the ability to bind a ligand. After binders are identified (thus, reducing the number of antigen-binding domains to be tested), another aliquot of each antigen-binding domain is combined with an effector domain, e.g., a glycosylated effector domain, and assayed, e.g., in a cell-based assay.

Of course, the same strategies can be applied to non-immunological target-binding domains and/or non-immunological effector domains. In any implementation, a target-binding domain bound to an effector domain by an interaction domain can also be covalently attached, e.g., by a disulfide bond or other crosslink within the interaction sequence or outside of this region.

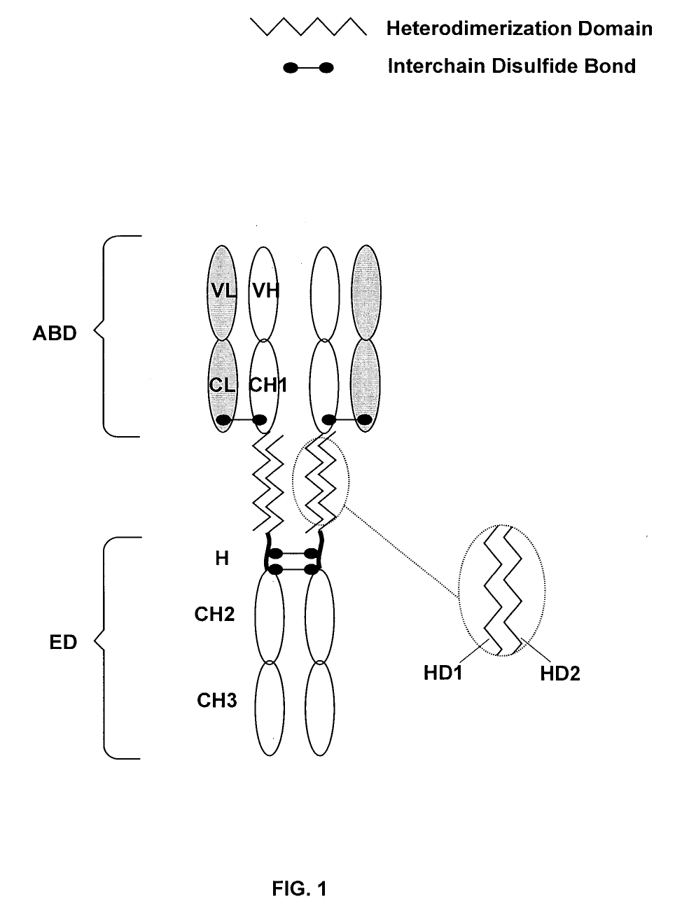

Referring to the example in FIG. 1, an exemplary protein includes two identical antigen-binding domains (ABD), which are Fab fragments, and an effector domain (ED). Each ABD is attached to an effector domain by a pair of heterodimerization sequences. One member (HD1) of each pair of heterodimerization sequence is attached to the Fab heavy chain; the other member (HD2) is attached to the dimeric Hinge (H)—CH2—CH3 effector domain.

The Fab fragments include the heavy chain fragment (VH—CH1) and the light chain (VL-CL) (not shown). The HD1 heterodimerization moiety can be attached to either the heavy chain fragment (as shown) or the light chain fragment (not shown).

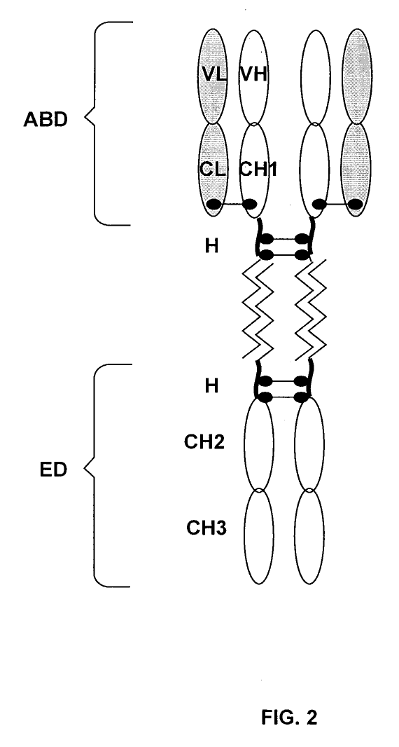

Referring to the example in FIG. 2, a protein can include two hinge regions (H), one on the ABD side, the other on the effector domain (ED) side. This construction recapitulates the proximity of the hinge region (H) to both the ABD and the ED.

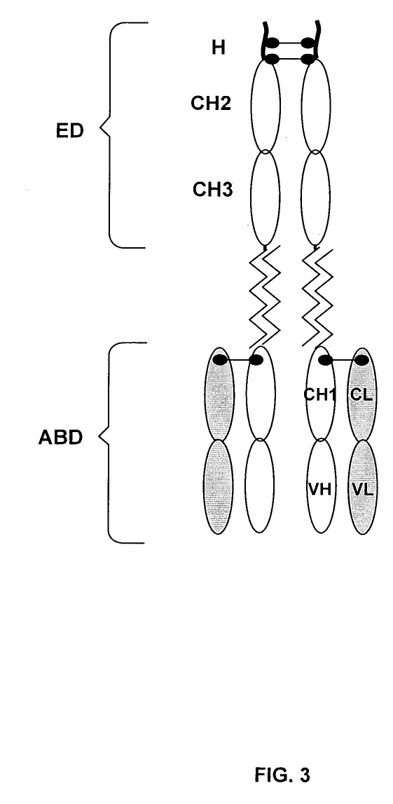

Referring to the example in FIG. 3, the ABD can include a single chain ABD, e.g., the scFV configuration in which the VH and VL domains reside in a single polypeptide chain.

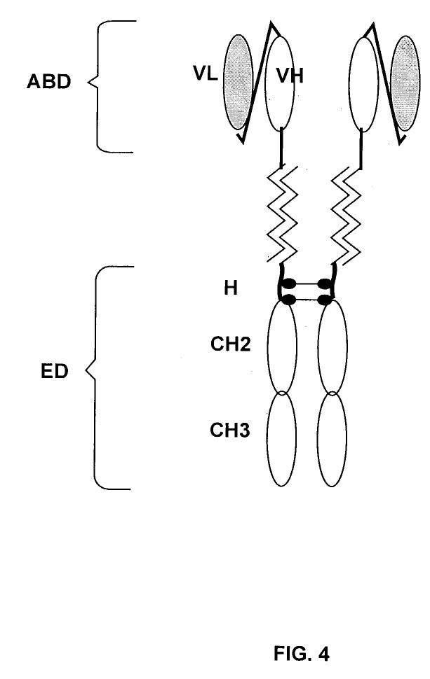

Referring to the example in FIG. 4, the heterodimerization sequence HD2 can be attached to the C-terminus of the effector domain. The hinge region is located at the N-terminus of the effector domain. Fab fragment ABDs are attached to HD2 by HD1-mediated heterodimerization.

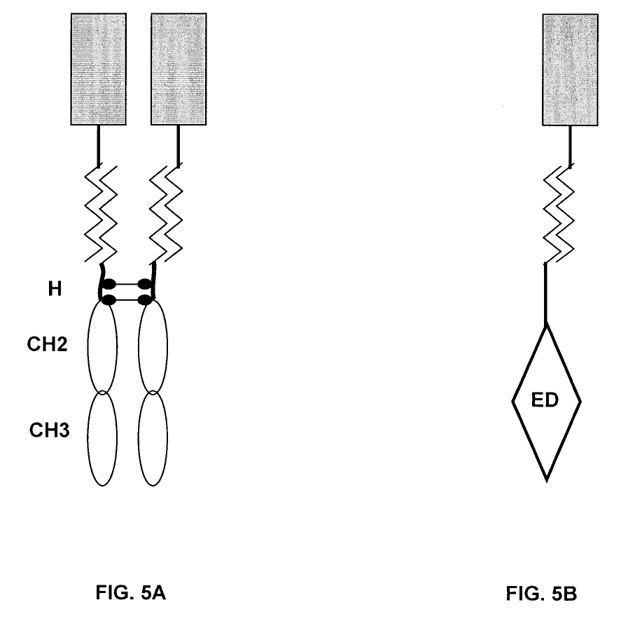

Referring to the example in FIG. 5A, two non-immunoglobulin target-binding domain are attached to an immunoglobulin effector domain by heterodimerization.

Referring to the example in FIG. 5B, a non-immunoglobulin target-binding domain is attached to a non-immunoglobulin effector domain (ED) by heterodimerization. For example, the target-binding domain can be a polypeptide hormone such as IL-2, and the effector domain can be a marker protein, e.g., green fluorescent protein.

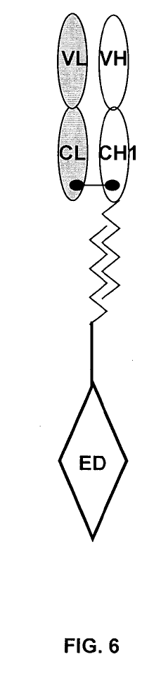

Referring to the example in FIG. 6, a Fab ABD is attached to a non-immunoglobulin effector domain by heterodimerization.

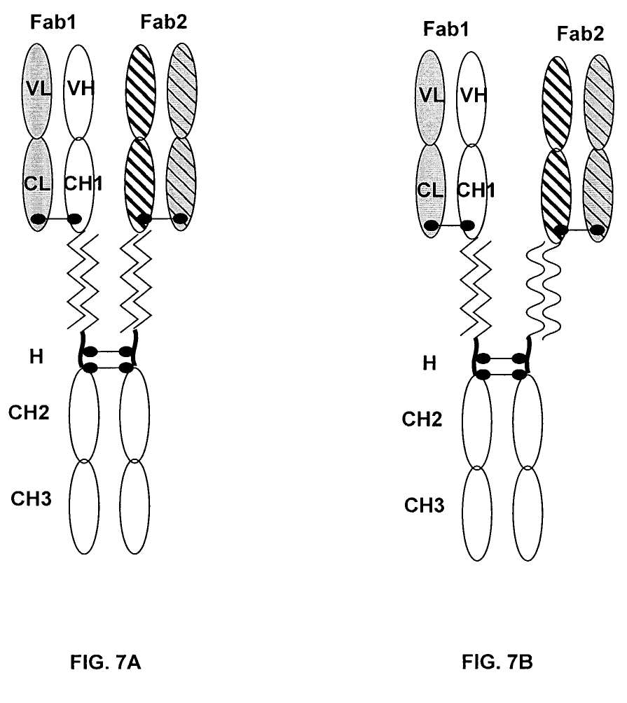

Referring to the example in FIGS. 7A and 7B, a bispecific protein is formed by heterodimerization of two ABDs (Fab1 and Fab2) and an immunoglobulin effector domain. In FIG. 7A, the bispecific protein is formed by combining a 1:1 mixture of Fab1 and Fab2 with the effector domain. Due to random assortment, about 50% of the complexes formed include Fab1, Fab2 and the effector domain.

In one embodiment, the mixture including the assorted complexes is used. If, for example, no undesired activity (such as undue competition) results from complexes of Fab1-Fab1-ED or Fab2-Fab2-ED, then the mixture can be used to provide (or detect) Fab1-Fab2-ED activity. In another embodiment, the desired complexes can be isolated, e.g., using appropriate purification tag. For example, the complexes are crosslinked in the heterodimerization region to prevent latter disassociation and re-assorting.

In FIG. 7B, two different heterodimerization pairs are used. For example, the fos-jun pair are used to heterodimerize Fab1 and the effector domain, whereas a pair of synthetic heterodimeric leucine zippers of different specificity than fos-jun are used to heterodimerize Fab2 and the effector domain. In this example, species of effector domains that include one chain having fos and the other chain having the synthetic zipper are purified. The two types of effector domain polypeptides can be co-expressed in the same cell and then isolated using affinity chromatography with a jun peptide column and then subsequent purification with the partner zipper of the synthetic zipper.

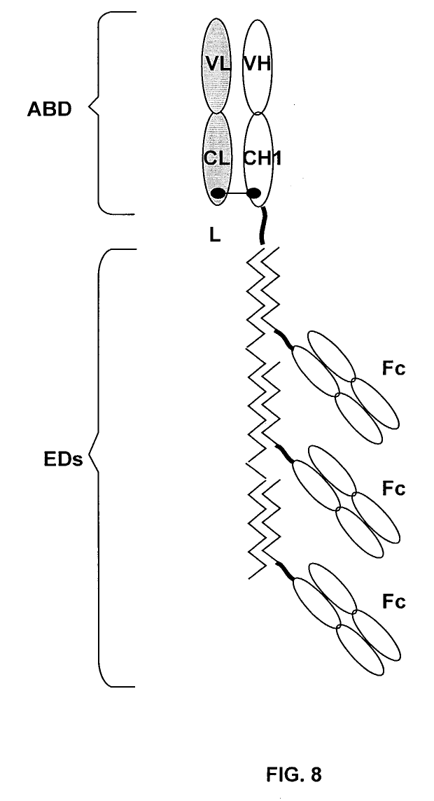

Referring to the example in FIG. 8, a Fab fragment is attached to multimerized heterodimerization sequences. The heterodimerization sequences are spaced from the Fab by a flexible linker (L). One effector domain is bound to each of the heterodimerization sequences in the multimer by a complementary heterodimerization sequence. As shown, the effector domains are dimeric Fc fragments in which one on the two chains is fused to the complementary heterodimerization sequence. Such asymmetric Fc fragments can be produced by coexpression of two of the appropriate immunoglobulin domains (i.e. Hinge-CH2—CH3 or CH2—CH3 for gamma-1), in which one is fused to one of the heterodimerization sequences, and purification of the heterodimer form of the Fc. Alternatively such asymmetric Fc fragments can be produced in more homogenous manners by methods described below, e.g., by “knobs-in-holes” engineering.

Target-Binding Domains

Antigen-binding domains. Antigen-binding domains typically include two immunoglobulin variables, e.g., the VH and VL variable domains. Each of these variable domains can include antigen binding residues located in or near three CDRs.

Antigen-binding domains can be obtained from a variety of sources.

In a first example, an antigen-binding domain is obtained from a monoclonal antibody. cDNA is prepared from mRNA isolated from the hybridoma that produces the monoclonal antibody. The genes encoding the monoclonal antibody's antigen-binding domain (i.e., VH—CH1 and VL-CL) are amplified from the genomic nucleic acid using the polymerase chain reaction and primers specific for conserved features in each chain. The amplified nucleic acids are cloned, e.g., into an expression vector.

In a second example, the antigen-binding domain is identified by screening a display library, e.g., a phage display library. Methods for screening antigen-binding domains using display libraries are described. See, e.g., U.S. Pat. No. 5,233,409; de Haard et al. (1999) J. Biol. Chem 274:18218-30; Hoogenboom et al. (1998) Immunotechnology 4(1):1-20. and Hoogenboom et al. (2000) Immunol Today 21(8):371-8).

In a third example, the antigen-binding domain is identified by immunization of an animal, e.g., a rodent and in some cases a transgenic rodent that includes a human immunoglobulin locus.

In a fourth example, both techniques are used, e.g., an antigen-binding domain obtained by immunization is further evolved using an in vitro mutagenesis technique.

Generally, an antigen-binding domain can be, for example, chimeric (e.g., including a variable domain from one species, and a constant domain from another), grafted (e.g., including a CDR from one species, and a FR from another species; see, e.g., U.S. Pat. No. 5,225,539), humanized (see, e.g., U.S. Pat. No. 5,585,089), deimmunized (see, e.g., WO 00/34317), or synthetic (e.g., a CDR encoding sequence is derived from a synthetic oligonucleotide).

Non-immunoglobulin target-binding domains. Other target-binding domains include an extracellular domains of proteins that can be to determinants on a cell, e.g., a prokaryotic or eukaryotic cell, preferably a mammalian cell. When a non-immunoglobulin target domain is coupled to an immunological effector domain such as Fc, the synthetic protein is termed an “immunoadhesin.”

Non-limiting examples of non-immunological target-binding domains include: peptide hormones, cytokines, extracellular matrix proteins, heterotypic cell adhesion molecules, viral proteins, bacterial attachment proteins, lectins, and T cell receptors. More particular examples of such domains include: retroviral glycoprotein ectodomains (HIV gp120 ectodomain, HTLV glycoproteins), influenza hemagglutinin, respiratory syncytial virus, papilloma virus surface proteins, chemokines (e.g., CCR4), CD4, CD8, CD52, platelet-derived growth factor; insulin-like growth factor-I and -II; nerve growth factor; fibroblast growth factor (e.g., aFGF and bFGF); epidermal growth factor (EGF); transforming growth factor (TGF, e.g., TGF-α and TGF-β); insulin-like growth factor binding proteins; erythropoietin; thrombopoietin;, interferon-α,β,γ; colony stimulating factors (CSFs), e.g., M-CSF, GM-CSF, and G-CSF; interleukins (ILs), e.g., IL-1, IL-2, IL-3, IL-4, etc.; decay accelerating factor; tumor necrosis factor (e.g., TNF-α and TNF-β); inhibin; activin; vascular endothelial growth factor, cell attachment molecules (“CAMs”) such as cadherins, selectins, N-CAM, E-CAM, U-CAM, and I-CAM.

Effector Domains

Effector domains are attached to an interaction sequence that can bind to the corresponding interaction sequence of the target-binding polypeptide chain. The interaction sequence can be attached to the N-terminus, C-terminus, or even internally. A linker or other flexible region can space the interaction sequence from the effector domain. Typically, the effector domain and the interaction sequence are translational fusions. However, the effector domain and the interaction sequence can also be attached by a non-peptide bond, e.g., a disulfide bond or other crosslink.

Fc domains. As discussed above, Fc domains mediate effector functions by recruiting C1q for complement-dependent cytotoxicity (CDC) and FcγRs for ADCC. In one embodiment, the effector domain is an Fc domain (CH2-CH3 and possible other domains if relevant for the particular antibody isotype) or an Fc domain and hinge region (H—CH2—CH3and possible other domains if relevant for the particular antibody isotype). In another embodiment, the effector domain includes a human gamma-1 Fc domain (CH2—CH3) or a human gamma-1 Fc domain and hinge region (H—CH2—CH3).

The Fc region of naturally-occurring IgG molecules is glycosylated at asparagine 297 in the CH2 domain. This asparagine is the site for modification with biantennary-type oligosaccharides. It has been demonstrated that this glycosylation is required for effector functions mediated by Fcγ receptors and complement C1q (Burton and Woof (1992) Adv. Immunol. 51:1-84; Jefferis et al. (1998) Immunol. Rev. 163:59-76). In one embodiment, the Fc domain is produced in a mammalian expression system that appropriately glycosylates the residue corresponding to asparagine 297. The Fc domain can also include other eukaryotic modifications.

The Fc domain can be attached to the hinge region, which is found between CH1 and CH2 of antibody heavy chains. The hinge region can impart a flexible structure that facilitates the recruitment of effector functions which bind in the CH2 domain in the proximity of the hinge region and also, e.g., antigen aggregation by a second antigen-binding domain. (See, e.g., Tan et al. (1990) Proc Natl Acad Sci U S A. 87:162-6.) Of course, in some embodiments, a flexible synthetic is used (see, e.g., Robinson and Sauer (1998) Proc Natl Acad Sci U S A.;95:5929-34). Flexible linkers can include glycine and hydrophilic amino acids such as serine.

In one embodiment, the Fc domain is a modified Fc domain. For example, the Fc domain can be altered, e.g., such that it has altered binding properties (e.g., enhanced or diminished). For example, the Fc domain can be engineered to preferentially binding to some Fc receptors relative to others. Shields et al. (2001) J Biol Chem 276:6591-6604 describes a variant IgG1 Fc domain that has improved binding to FcγRIIIA. Idusogie et al. (2000) J. Immunol. 164:4178 describes an IgG1 mutant that alters C1q binding and complement activation.

In still another embodiment, the effector domain is a synthetic polypeptide that binds to an Fc receptor or to complement. Such synthetic polypeptides can be identified by a phage display selection for 6 to 20 amino acid cyclic peptides that specifically binding to one species of Fc receptor, but not another.

Other types of polypeptide and polypeptide conjugates can be used as an effector domain.

Labels. For example, the effector fragment can include a polypeptide label or a non-polypeptide label. Polypeptide labels include enzymes, such as horseradish peroxidase, alkaline phosphatase, β-galactosidase, or acetylcholinesterase. Other polypeptide labels include luciferase, luciferin, aequorin, and green fluorescent protein (and its derivatives). For example, an effector domain fragment that includes GFP can be used to identify the localization of a target in a sample, e.g., a histological sample.

A polypeptide conjugate can also be used as an effector domain. In this embodiment, a peptide is synthesized (chemically or in cells) to include an interaction sequence (e.g., c-fos) and a chemical linker for a chemical group. Examples of chemical labels include umbelliferone, fluorescein, fluorescein isothiocyanate, rhodamine, dichlorotriazinylamine fluorescein, dansyl chloride or phycoerythrin. Other labels that can be attached include a radioactive nucleus such as ¹²⁵I, ¹³¹I, ³⁵S or ³H, or an imaging agent, e.g., a NMR contrast agent.

The effector fragment can include a single free cysteine in addition to the interaction domain. The free cysteine can be used to attach a non-peptide effector agent using thiol chemistry.

Synthetic peptides can include a single cysteine to which a label or other chemical compound can be attached.

Cytotoxins. Polypeptide and non-polypeptide cytotoxins can be used as an effector domain. Examples of polypeptide cytotoxins include diphtheria toxin, cholera toxin, abrin, pseudomonas exotoxin, and ricin A. Non-polypeptide cytotoxins can be chemically coupled to the compatible interaction sequence, e.g., as described above. Examples of non-polypeptide cytotoxins include: taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, and analogs or homologs thereof.

Other Effector Domains. The effector fragment can also include other domains, e.g., domains with a therapeutic or cell-signaling function. Examples of effector domains with signaling functions include tumor necrosis factor, interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Interaction Domains

In some embodiments, compatible interaction sequences are used to attach a target-binding domain to an effector domain. Typically, the resulting attachment is non-covalent. However, in some implementations, cysteines or other reactive residues are positioned in sufficient proximity that a covalent bond is formed.

The compatible interaction sequences can be members of a binding pair, e.g., a specific binding pair can be used. Typically, for binding pairs, the two proteins form a heterodimer, e.g., a third polypeptide is not required to mediate the interaction. The two proteins can have an affinity for each other of less than 100 nM. One example of a heterodimeric interaction pair is a coiled-coil such as a leucine zipper.

Leucine Zippers. Leucine zippers are amino acid sequences of about 20-40 residues long in which leucine typically occurs every seventh residue (Landschulz et al. (1988) Science 240:1759). The amino acid sequence of leucine zippers can be summarized as follows:

(L-X₆)ₙ (I)

where L is leucine and X is any amino acid, but preferably not cysteine. n can be 3 or more, e.g., about 4 or 5. In a preferred embodiment, the fourth position of each repeat is also hydrophobic, e.g., an aliphatic amino acid.

The hydrophobic leucines are packed into a central core of a dimeric structure formed from two pairing leucine zippers. Other residues determine other parameters include stability, specificity, and monomeric state. For example, some leucine zipper sequences form trimers.

One pair of preferred leucine zipper proteins includes the proto-oncogenes c-fos and c-jun. c-fos and c-jun are transcription factors that form the heterodimeric AP-1 complex that drive expression of some mammalian genes. As used herein “fos” refers to the c-fos leucine zipper set forth in SEQ ID NO: 1, a variant thereof with no more than 5 mismatches, or a permuted variant. As used herein “jun” refers to the c-jun leucine zipper set forth in SEQ ID NO: 2, a variant thereof with no more than 5 mismatches, or a permuted variant.

The fos and jun leucine zippers have been demonstrated to preferentially form heterodimers rather than homodimers (O'Shea et al. (1989) Science 245:646). For example, the two peptides can form a population of dimers that is greater than 50% heterodimeric, more commonly greater than 85% heterodimeric. It is also known that the leucine zipper regions of these two protein alone are sufficient to mediate heterodimerization. Both are relatively short polypeptides—less than 45 amino acids in length—and both are devoid of cysteines.

Other preferentially heterodimeric leucine zippers are described in Arndt et al. (2000) J. Mol. Biol. 295:627. Moreover, other heptad repeat coiled-coils in which a hydrophobic residues appears every seven residues can be used.

Other Specific Binding Pairs. U.S. Pat. No. 6,294,353 describes the use of different segments of a single folded protein unit to mediate interactions. Association of the different segments reforms the folded unit. For example, the segments can be segments of an enzyme. Association of the two segments can form a folded and functional enzyme. Still other specific binding pairs include natural proteins and their ligands, e.g., calmodulin and a calmodulin-binding protein, e.g., calbindin. Protein engineering can also be used to modify dimeric proteins so that they are heterodimeric. See, e.g., Nohaile et al. (2001) Proc. Natl. Acad. Sci. USA 98:3109-14 and Hendsch et al. (2001) J Am Chem Soc. 123:1264-5.

Extracellular Interaction Domains. In one embodiment, the interaction domains are domains of extracellular proteins that interact. Examples of extracellular interaction domains include: Notch and Delta ectodomains, heterotypic cell adhesion molecules, and integrin α and β subunits.

Bridged Interaction Domains. In another embodiment, the inter-molecular interaction is bridged by a moiety, e.g., a peptide or non-peptide moiety. For example, Lin and Cornish (2001) Angew. Chem. Int. Ed. 40:871 describe chemical inducers of heterodimerization that include two linked chemical ligands, e.g., dexamethasone linked to FK506, which can be used to heterodimerize a hormone binding domain and FKBP, the FK506 binding protein.

Modeling. For some applications, the configuration of interaction domains and other components (including, e.g., antigen-binding domains, effector domains, and linkers) are designed and modeled using a computer (see, e.g., Ewing et al. (2001) J Comput Aided Mol Des 15:411-28; U.S. Pat. No. 4,946,778). Software for molecular modeling is commercially available (e.g., from Molecular Simulations, Inc.). Modeling efforts can be directed to determining if obvious steric or flexibility issues may interfere with the function and/or structure of the designed molecule.

Multimerized Interaction Domains

Multimers of one or both compatible interaction domains (typically members of a specific binding pair) can be used to alter the ratio of target binding and effector domains. For example, a single target-binding domain can be coupled to multiple effector domains by attachment of multimers of an interaction sequence.

Referring again to FIG. 8, a Fab fragment is attached to multiple effector domains by a multimer of heterodimerization sequences. Each of the heterodimerization sequences in the multimer is bound by a complementary heterodimerization sequence that is connected to an effector domain. The heterodimerization sequences within the multimer preferentially do not interact with each other.

This multimer configuration can emulate effector domain aggregation or enhance effector mechanisms. In another implementation, different effector domains are attached by including domains of differing specificities in the multimer. Conversely, multiple target-binding domains can be attached to an effector domain by connecting effector domains to multimerized heterodimerization sequence, and then attaching a target-binding domain with the compatible heterodimerization sequence. Different target-binding domains can be attached if the heterodimerization sequences in the multimer have differing specificities.

Of course, large macromolecular assemblies can be constructed by including multimerized interaction sequences on both the target-binding and effector domains.

Asymmetric Fc Proteins

An asymmetric Fc protein can be produced using “knobs-into-holes” engineering, e.g., as described by Ridgway et al. (1996) Protein Eng 9:617-21. To produce an Fc region that includes a single interaction domain, two different Fc polypeptides are expressed in a cell, e.g., a eukaryotic cell. One polypeptide includes the interaction sequence and, for example, knobs in the CH3 region, such as T366Y. The other polypeptide lacks the interaction sequence and includes the “hole” such as Y407T. Co-expression of the two polypeptides can result in a large proportion of asymmetric Fc proteins. Small populations of symmetric Fc proteins can be removed, e.g., by chromatography. However, such removal may not be necessary for at least some implementations.

Polypeptide Production

In one embodiment, the antigen-binding fragment is produce in bacterial cells, e.g., E. coli cells. For example, nucleic acids encoding the one or more chains of an antigen-binding fragment are cloned into a prokaryotic expression vector. The nucleic acids are fused in frame with an N-terminal signal sequence that directs secretion of the downstream polypeptide sequence. In the case of a multi-chain antigen-binding fragment (e.g., a Fab), the nucleic acids encoding each chain can be expressed from the same or different plasmids. In one embodiment, the nucleic acids are linked in tandem in an operon, e.g., an operon regulated by an inducible promoter. The bacterial host cells are cultured and induced. After induction, cells can be isolated. A periplasmic-shock fluid can be prepared to release the secreted antigen-binding fragment from the periplasm. See, e.g., Ausubel et al., Current Protocols in Molecular Biology (2001) Greene Publishing Associates and Wiley Interscience, N.Y. and associated on-line resources. If one of the polypeptide chains includes a purification tag, the antigen-binding fragment can be purified using affinity chromatography based on the tag. Conventional chromatographic methods can also be used. See, e.g., Scopes (1994) Protein Purification: Principles and Practice, New York: Springer-Verlag.

As appropriate, effector domains can also be produced in bacterial cells.

In a preferred embodiment, the effector fragment includes an extracellular domain, e.g., an Fc domain, and is produced in eukaryotic cells, e.g., mammalian cells or yeast cells. Exemplary mammalian host cells include Chinese Hamster Ovary (CHO cells) (including dhfr-CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NS0 mycloma cells and SP2 cells, COS cells, and a cell from a transgenic animal, e.g., a mammary epithelial cell.

In an exemplary system for recombinant expression of an effector fragment, a nucleic acid sequence encoding the effector fragment and a interaction sequence is inserted into an expression vector. The sequence can be operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The expression vector is introduced into dhfr-CHO cells, e.g., by calcium phosphate-mediated transfection. The selected transformant host cells are culture to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. For example, the Fc domain can be isolated by affinity chromatography with a Protein A.

In another embodiment, the target-binding fragment (e.g., an antigen-binding fragment or a non-immunoglobulin target-binding fragment) is expressed in a mammalian cell, e.g., using a method described herein or another method.

Purification Handles

In some embodiments, the target-binding fragment or the effector polypeptide includes a purification handle in order to facilitate isolation of these polypeptides from an expression system. The purification handle is one component of a specific binding pair.

One purification handle is the hexa-histidine tag (see, e.g., German Patent No. DE 19507 166). This moiety binds avidly to Ni²⁺ NTA (nitrilotriacetic acid ) and can be eluted under mild conditions with imidazole. Other specific binding pairs include the following: glutathione-S-transferase and glutathione; chitin binding protein and chitin; cellulase (CBD) and cellulose; maltose binding protein and amylose and or maltose; dihydrofolate reductases and methotrexate; and FKBP and FK506.

Another class of specific binding pair is a peptide epitope and the monoclonal antibody specific for it (see, e.g., Kolodziej and Young (1991) Methods Enz. 194:508-519 for general methods of providing an epitope tag). Exemplary epitope tags include HA (influenza hemagglutinin; Wilson et al. (1984) Cell 37:767), myc (e.g., Myc1-9E10, Evan et al. (1985) Mol. Cell. Biol. 5:3610-3616), VSV-G, FLAG, and 6-histidine.

Protein Verification

The integrity of a protein composition described herein can be monitored using standard techniques of protein chemistry. For example, after combining an antigen-binding fragment linked to a first heterodimerization sequence and an effector domain linked to a second heterodimerization sequence, the complex can be subjected to column chromatography, e.g., gel exclusion chromatography (although in some cases ion exchange chromatography is also applicable). Fractions from the separation are collected and analyzed to determine if the antigen-binding fragment and the effector fragment elute in the same fraction. The approximately molecular weight of the complex can be estimated from the fraction number using calibrated size standards.

In another example, the molecular weight of the complex (if mono-disperse) is analyzed by equilibrium ultracentrifugation. In still another example, the complex is analyzed by precipitating the complex using a ligand that is specific for the antigen-binding fragment or the effector fragment. Either or both of these fragments can have purification tags. Using a ligand attached to a solid support, the fragment with the tag can be separated from the solution. If stable complexes are formed, then the fragment without the tag is also separated by virtue of the interaction between the two fragments. The fraction of complexed fragments can be determined or estimated based on the separation.

Further, an ELISA assay can be used test the integrity of the complex. Wells of a microtitre plate are coated with the antigen that is recognized by the antigen-binding fragment. Wells are washed and coated with a non-specific blocking agent. Then, the wells are contacted with different concentrations of the complex and washed extensively. Then the amount of effector fragment bound to each well is quantitated using an enzyme-linked probe that is specific for the effector fragment, e.g., an antibody that recognizes the effector fragment.

Screening Methods

The separation of target-binding domain and effector domain into separate polypeptides facilitates the high-throughput screening (including automated screening) of target-binding domains, e.g., from an expression library such as a cDNA library or a display library.

One exemplary screen includes screening antigen-binding domains, e.g., Fab's and scFv's. Other target-binding domains (e.g., synthetic peptides and modified scaffold domains) can be similarly screened.

The antigen-binding domains are first screened for a binding property. Phage display is one convenient format for identifying polypeptides with a desired binding property. Binding can also be verified using an ELISA assay, e.g., while the antigen-binding domain is displayed on a phage.

The antigen-binding domain can be composed of a heavy chain fragment, e.g., VH and CH1 domains, and a light chain, e.g., VL and CL. Nucleic acids expressing these polypeptides can be into a bacterial expression vector that includes features for filamentous bacteriophage display and for heterodimerization. A plasmid for expressing the polypeptide that is attached to the interaction sequence (e.g., a heterodimerization) and the phage coat includes one or more of the following nucleic acid sequences: an insert segment (e.g., a polylinker, the light chain, the heavy chain or other derivative, e.g., scFv), a heterodimerization sequence, a purification tag, a suppressible stop codon, and a site for attachment to the phage particle (e.g., a fusion to the gene III protein or fragment thereof).

The antigen-binding domain can be presented on the surface of a filamentous phage by transferring the phagemid into bacterial host cell that has a tRNA suppressor gene so that when the plasmid insert is expressed, the polypeptide encoded by the insert is secreted and attached (e.g., fused) to both the interaction sequence and the phage particle. The other chain of the antigen-binding domain is likewise expressed and secreted. The host cell is also infected with a helper bacteriophage, e.g., VCSM13, to produce infectious bacteriophage that harbor the phagemid and display the antigen-binding domain.

The phagemid is recovered from the bacteriophage, e.g., after selection and amplification. The phagemid is then transformed into a non-suppressing bacterial host cell such that when the plasmid insert is expressed (e.g., by induction), the cells secrete a polypeptide that includes the amino acid sequence encoded by the insert and the interaction sequence, but is no longer attached to the phage particle. Again, the other chain of the antigen-binding domain is also expressed and secreted by the same host cell. The two chains associate in the periplasm to produce a soluble antigen-binding domain that includes an interaction sequence. If a purification handle is also present, then the antigen-binding domain can be easily purified using affinity chromatography.

These steps can be performed for a large number of different phagemids, each encoding a different antigen-binding domain. The transfer to a non-suppressing bacterial host cell and the purification of soluble antigen-binding domain can be automated or semi-automated. The purified antigen-binding domain is then combined with an effector fragment that includes the effector domain and an interaction sequence that specifically binds the corresponding interaction sequence on the antigen-binding fragment. The effector domain can be the Fc domain, for example, produced in mammalian cells, e.g., by fermentor production.

The mixtures of bacterially-produced antigen-binding domain and mammalian cell-produced Fc domain are individually assayed for functionality, e.g., as described below.

In another related embodiment, after identification of an antigen-binding domain in a phage display library, the phage displaying the identified antigen-binding domain are themselves used in a function assay. As displayed, the phage present the antigen-binding domain and the interaction sequence outside of the phage coat. The phage are contacted (before, during, or after binding to the target) with effector domain that includes a corresponding interaction domain. The effector domain can then recruit effector functions such as cytotoxic T cells. In one embodiment, the interaction sequence attached to the antigen-binding domain is multimerized so that multiple effector domains are recruited, thus increasing the aggregation of effector domains by the antigen-binding domain on the target. Aggregation is required for at least some effector functions.

In yet another embodiment, the target-binding domains are released from the phage particles by a chemical or enzymatic treatment. The released domains, which still include an interaction domain, are contacted with the effector domain that includes a corresponding interaction domain.

Functional Assays

The proteins formed by heterodimerization of antigen-binding domain and effector domain as well as other complexes (covalent and non-covalently formed) described here can be assayed for functional activity either in vitro or in vivo.

In vitro assays include assays for immunoglobulin effector domain activity, e.g., cytotoxic activity. For example, cell culture assays can be used to assay complement dependent cytotoxicity (CDC) or antibody-dependent cell-mediated cytotoxicity (ADCC). One ADCC assay is described below.

The Cr-release assay, for example, can be used to assay cell-mediated cytotoxicity. Peripheral blood lymphocytes (PBL) are prepared as effector cells, while target cells that express the targeted MHC-peptide complex are loaded with ⁵¹Cr. The target cells are washed and then seeded into a flat bottom microtitre plate. PBLs are added to the target cells in combination with the ligand (e.g., a known anti-(MHC-peptide complex) ligand or a candidate ligand). Maximum release is determined by the addition of Tween-20 to target cells, whereas minimal release is determined in the absence of PBLs. After overnight incubation, ⁵¹Cr released into the supernatant is counted in a γ scintillation counter.

In vivo assays include injecting a protein complex (covalent and non-covalently formed as described herein) to an animal, e.g., an animal model of a diseased state. If the effector domain being assayed is an Fc region, then a species-compatible Fc region can be used. For example, to test a human Fab as the target-binding domain in a mouse, the murine Fc region can be used. The animal used for the assays can be a transgenic animal, e.g., an animal expressing an oncogene in a particular tissue. In another example, the animal is infected with a virus or other pathogen. In another example, the animal is a mouse with a xenograft of human tumor cells. The test mouse can be a standard laboratory mouse, a transgenic mouse that is unable to make murine antibodies, or a nude mouse. For example, a nude mouse can be supplied with human lymphocytes.

The efficacy of the protein complex can be assayed by comparing time, size, and number of tumors formed compared to untreated or control-treated animals. One useful control is one or a subset of components of the protein complex, e.g., the antigen-binding fragment without the effector fragment, or the effector fragment without the antigen-binding fragment. Other physiological parameters can also be monitored including immunogenicity, clearance, and so forth.

Effector Domain Screen

In one embodiment, the separation of target-binding and effector functions into different polypeptides is applied to a screen for effector domains. Such a screen includes providing a given target-binding domain that specifically recognizes a target molecule, and a plurality of candidate effector domains. Each candidate effector domain is attached to the target-binding domain and then tested for effector activity, e.g., by contacting to a target cell that includes the target molecule and an effector cell.

The candidate effector domains can be, for example, members of a cDNA library, a library of diversified scaffold domains (such as a Fab or a Kunitz domain), a library of synthetic peptides, and so forth. Typically, the candidate effector domains are preselected members of such a library. For example, a phage display library can be screened to identify ligands that bind to an FcγRI protein. Ligands identified in the library can be synthesized with a heterodimerization sequence and then attached to the target-binding domain that includes a complementary heterodimerization sequence. The ability of the ligands to evoke ADCC against a target cell can be tested in vitro and/or in vivo.

In a similar respect, a library-against-library screen can be implemented. The first library is a library of target-binding proteins and the second library is a library of effector proteins. Each combination of target-binding domain and effector domain is tested by joining the two domains using a method described herein. The libraries can either be, e.g., a pre-selected collection of proteins or an unsampled collection.

In one embodiment of a library-against-library screen using secretion vectors, each library is constructed in a population of yeast cells. The target-binding domain library is introduced into yeast cells of a first mating type, whereas the effector domain library is introduced into yeast cells of a second mating type. Combinations of each member of the first and second library are formed by mating the cells of the respective libraries. Each mated yeast cell thus formed secretes a protein complex that includes the target-binding protein and the effector protein combinations.

In another embodiment of the library-against-library screen, proteins are purified from each library member. Combinations are produced by combining aliquots of a targeting binding protein and an effector domain protein.

Automated Screening

The methods and compositions provided herein are also suitable for automated screening of diversity libraries. For example, a display library of Fab's can be screened for members that bind to a target molecule. Binders from a first round of screening can be amplified and rescreened, one or more times. Binders from the second or subsequent rounds are individually isolated, e.g., in a multi-well plate. Each individual binder can then be assayed for binding to the target molecule, e.g., using ELISA, a homogenous binding assay, or a protein array. These assays of individual clones can be automated using robotics. Results of the assay can be stored in a computer system and evaluated using software, e.g., to identify clones which meet particular parameters (e.g., for binding affinity and/or specificity).

A robotic apparatus can be directed to manipulate the nucleic acid of the identified clones to synthesize the proteins encoded by the clones. The synthesized proteins are then attached covalently (e.g., using protein ligation or crosslinking) or non-covalently (e.g., using heterodimerization sequences) to an effector domain. The attached proteins can then be assayed for a functional property that depends on the effector domain (See, e.g., “Functional Assays,” above). These assays can also be automated, and their results stored and/or processed to identify useful members of the diversity library.

The following describes possible embodiments of exemplary assays for binding assays:

ELISA. Polypeptides encoded by a display library can also be screened for a binding property using an ELISA assay. For example, each polypeptide is contacted to a microtitre plate whose bottom surface has been coated with the target, e.g., a limiting amount of the target. The plate is washed with buffer to remove non-specifically bound polypeptides. Then the amount of the polypeptide bound to the plate is determined by probing the plate with an antibody that can recognize the polypeptide, e.g., a tag or constant portion of the polypeptide. The antibody is linked to an enzyme such as alkaline phosphatase, which produces a colorimetric product when appropriate substrates are provided. The polypeptide can be purified from cells or assayed in a display library format, e.g., as a fusion to a filamentous bacteriophage coat. In another version of the ELISA assay, each polypeptide of a library is used to coat a different well of a microtitre plate. The ELISA then proceeds using a constant target molecule to query each well.

Homogeneous Binding Assays. The binding interaction of candidate polypeptide with a target can be analyzed using a homogenous assay, i.e., after all components of the assay are added, additional fluid manipulations are not required. For example, fluorescence resonance energy transfer (FRET) can be used as a homogenous assay (see, for example, Lakowicz et al., U.S. Pat. No. 5,631,169; Stavrianopoulos, et al., U.S. Pat. No. 4,868,103). Another example of a homogenous assay is Alpha Screen (Packard Bioscience, Meriden Conn.). Alpha Screen uses two labeled beads. One bead generates singlet oxygen when excited by a laser. The other bead generates a light signal when singlet oxygen diffuses from the first bead and collides with it. The signal is only generated when the two beads are in proximity. One bead can be attached to the display library member, the other to the target. Signals are measured to determine the extent of binding. The homogenous assays can be performed while the candidate polypeptide is attached to the display library vehicle, e.g., a bacteriophage.

Protein Arrays. Polypeptides identified from the display library can be immobilized on a solid support, for example, on a bead or an array. For a protein array, each of the polypeptides is immobilized at a unique address on a support. Typically, the address is a two-dimensional address. Methods of producing polypeptide arrays are described, e.g., in De Wildt et al. (2000) Nat. Biotechnol. 18:989-994; Lueking et al. (1999) Anal. Biochem. 270:103-111; Ge (2000) Nucleic Acids Res. 28, e3, I-VII; MacBeath and Schreiber (2000) Science 289:1760-1763; WO 01/40803 and WO 99/51773A1. Polypeptides for the array can be spotted at high speed, e.g., using commercially available robotic apparati, e.g., from Genetic MicroSystems or BioRobotics.

Covalent Linkages

The target-binding fragment and the effector fragment can be produced separately and then covalently linked after synthesis. For example, the two fragments can be crosslinked or ligated together as described below. Covalently linked molecules can be administered as therapeutics or used in assays, e.g., screening assays.

Crosslinking. One type of compound is produced by crosslinking a target-binding fragment to an effector fragment. These fragments may or may not include compatible interaction domains. Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Crosslinks can also be constructed from free cysteines on the antigen-binding fragment and on the effector polypeptide. For example, if the heterodimerization sequences of these polypeptides are fos and jun, a free cysteine can be positioned at the carboxy terminus of each, e.g., spaced by two to four glycines from the last position of the leucine zippers. After heterodimerization, a disulfide bond can be formed between the two polypeptides by virtue of the free cysteines. Optionally, the two polypeptides are subjected to mild reducing conditions, e.g., prior to complex formation or prior to oxidation to form the disulfide. The cysteines can also be placed within the leucine zippers at positions compatible with crosslinking.

Inteins. A target-binding fragment and an effector domain can be joined by protein ligation, e.g., intein-mediated protein ligation. PCT WO 00/47751 describes a method of protein ligation. The method allows two separately synthesized polypeptide domains to be covalently joined with a peptide bond. The two domains are produced separately. Each is produced as a fusion to a different variant of an intein, e.g., the Methanobacterium thermotrophicum RIR1 intein. The domain that is intended for the N-terminus of the resultant protein is expressed as a polypeptide fusion of the N-terminal domain itself, a C-terminal variant intein domain and then a purification tag. For example, the intein variant is the P⁻¹G/N¹³⁴ A variant of RIR1 described in WO 00/47751. After purification using the tag, the N-terminal domain can be activated by treatment with 100 mM 2-mercaptoethanesulfonic acid (MESNA) to generate a thioester at the C-terminus.

The domain that is intended for the C-terminus of the resultant protein, e.g., the effector fragment, is expressed as a polypeptide of a N-terminal purification domain, an N-terminal intein variant, e.g., the P⁻¹G/C¹ A mutant of RIR1 described in WO 00/4775 1, and then the C-terminal domain itself. The polypeptide is incubated in a buffer of neutral pH (e.g., about 7.0) and the C-terminal domain is released with an N-terminal cysteine.

The released C-terminal domain, which includes an N-terminal cysteine, is combined with the released N-terminal domain, which includes a reactive thioester at its C-terminus under conditions that favor ligation of the two domains. Protein ligation can be monitored, e.g., by gel electrophoresis.

Hence, the invention also features a polypeptide chain that includes an immunoglobulin domain and an intein (e.g., a C-terminal intein) that can be modified to form a C-terminal thioester, e.g., by MESNA. Further, the invention features a method for covalently joining two an antigen-binding fragment and an effector polypeptide. The method includes providing a first polypeptide that includes an antigen-binding domain (e.g., an immunoglobulin variable domain) and a first variant intein and a second polypeptide that includes an effector domain and a second variant intein; cleaving the first variant intein from the first polypeptide to yield a modified first polypeptide that has a thioester at its carboxy terminus; cleaving the second polypeptide to yield a modified second polypeptide that includes a cysteine at its N-terminus; and ligating the modified first polypeptide to the modified second polypeptide. The first and second polypeptide can be expressed in different cells, e.g., a prokaryotic cell and a mammalian cell.

Cell-Attachment

The bridging and covalent attachment methods can also be used to attach a target-binding domain to a cell surface protein on a cell, e.g., an effector cell. The cell surface protein can include, e.g., a transmembrane domain or other linkage to the plasma membrane (e.g., a phosphoinositol linkage).

In one example, a Fab fragment that includes an interaction sequence is contacted to any effector cell (T-cell, Natural Killer cell, dendritic cell, Macrophage, Neutropil) cell that expresses a cell surface protein that includes a compatible interaction sequence positioned in the extracellular region. This latter may be a naturally occurring interaction sequence, a domain provided for by chemical modification of the cell surface, or a domain expressed from a transgene introduced into the cell (or parent thereof). The compatible interaction sequences interact to attach the Fab to the cell. The Fab can be used to direct the effector mechanism of the cell (i.e. T-cell activation, T-cell help, CTL-mediated cellular cytotoxicity, NK cell mediated cytotoxicity, antigen uptake, etc.) against particular targets and/or target cells.

The target-binding domain and its interaction sequence is contacted to the cell in vivo or in vitro. The cell can then deliver an effector function.

In one embodiment, a transgenic mouse is constructed that includes a transgene for that encodes a polypeptide that includes a transmembrane domain and an interaction sequence on a cell plasma membrane. The transgene can include a regulatory sequence for expressing the heterologous polypeptide in particular cell types (e.g., immune cells). The target domain and its compatible interaction sequence can be administered to the mouse and the mouse assayed for effector functions.

Additional Applications

The bridging of at least two polypeptide chains by interaction domains can overcome certain problems with the direct fusion of proteins that require their N- or C-terminus for activity or structural stability. The interaction domains can be positioned at the terminus that is not susceptible and the other polypeptide can be attached by heterodimerization. For example, both an antigen-binding domain and an Fc domain can be coupled to complementary interaction domains at their C-termini.

In one implementation, the interaction domain on an effector domain or target-binding domain can be used to attach such a domain to a solid support that has attached a compatible interaction domain. In the case of a screen, this strategy facilitates the construction, for example, of a protein array including the different proteins being screened. In one implementation, each screened protein is purified. One aliquot of the protein preparation is attached to an address of a protein array while another aliquot is used to attach the screened domain to another domain (e.g., a target-binding domain to an effector domain, and vice versa) to form a bifunctional protein.

The following example is merely illustrative of particular aspects of the invention described herein.

### EXAMPLE 1

One c-fos zipper is: LQAETDQLEDEKSALQTEIANLLKEKEKL (SEQ ID NO: 1).

One c-Jun zipper is LEEKVKTLKAQNSELASTANMLREQVAQL (SEQ ID NO: 2).

Longer forms of these zippers are as follows:

c-fos: LTDTLQAETDQLEDEKSALQTEIANLLKEKEKLEFILA (SEQ ID NO: 3).

c-Jun: RIARLEEKVKTLKAQNSELASTANMLREQVAQLKQKVMN (SEQ ID NO: 4).

A first nucleic acid is constructed that encodes, from the N-terminus to the C-terminus, the VH and CH1 domains of the heavy chain of a particular antibody, the c-fos zipper, and a hexa-histidine tag. The first nucleic acid is expressed in the same bacterial cell as a second nucleic acid encoding the antibody light chain. Fab fragments are purified from the periplasm of the bacterial cell after a suitable induction period.

A third nucleic acid is constructed that encodes, from the N-terminus to the C-terminus, a hexa-histidine tag, the c-jun zipper (SEQ ID NO: 2), the hinge domain, the CH2 and CH3 domains of IgG1. This nucleic acid is expressed in a mammalian cell. The effector domain is purified from the mammalian cell or media.

The purified Fab and the effector domain are combined, and then tested for cell-mediated cytotoxicity against cells that express the antigen.

### EXAMPLE 2

Alternative c-Jun zippers are used. These zippers have reduced ability to form homodimers, but still heterodimerize with c-Fos (Smeal et al. (1989) Genes & Development 3:2091-2100).

Some c-Jun zippers with reduced heterodimerization ability include:

| | |
|---|---|
| LEEKVKTLKAQNSELASTFNMLREQFAQL; | (SEQ ID NO:5) |
| LEEKVKTLKAQNSELASTANMLREQVAQF; | (SEQ ID NO:6) |
| LEEKVKTFKAQNSELASTANMLREQVAQF; | (SEQ ID NO:7) |
| LEEKVKSFKAQNSEHASTANMLREQVAQL. | (SEQ ID NO:8) |

A number of embodiments of the invention have been described. Nevertheless, it will be understood that various modifications may be made without departing from the spirit and scope of the invention. Accordingly, other embodiments are within the scope of the following claims.

What is claimed is:

1. A protein comprising:
a first polypeptide that includes a first immunoglobulin domain and a first interaction sequence, wherein the first interaction sequence specifically recognizes a second interaction sequence; and
a second polypeptide that includes the second interaction sequence and an effector domain that does not include an immunoglobulin variable domain.

2. The protein of claim 1 wherein the first immunoglobulin domain comprises a VH or VL domain.

3. The protein of claim 1 wherein the first polypeptide further comprises a second immunoglobulin domain.

4. The protein of claim 1 further comprising a third polypeptide that includes a second immunoglobulin domain.

5. The protein of claim 1 wherein the effector domain comprises CH2 and CH3 domains.

6. The protein of claim 1 wherein the effector domain is glycosylated.

7. The protein of claim 5 wherein the effector domain is glycosylated on at least an asparagine corresponding to asparagine 297 of CH2.

8. The protein of claim 1 wherein the first polypeptide is synthesized in vitro or in a bacterial cell and the second polypeptide is synthesized in a mammalian cell.

9. The protein of claim 1 wherein the first and second interaction sequences are components of a coiled-coil.

10. The protein of claim 1 wherein the first polypeptide comprises a multimer of interaction sequences, one of which is the first interaction sequence.

11. A compound comprising:
a first polypeptide that includes at least a part of a first target-binding domain and a first interaction sequence; and
a second polypeptide that includes the second interaction sequence and at least a part of an effector domain; wherein the first interaction sequence can bind to the second interaction sequence and the effector domain is has one or more of the following properties: a) binds (e.g., specifically binds) to a surface of a cell, b) is functional in an extracellular environment, or c) is a detectable label (i.e., other than being antigenic).

12. The compound of claim 11 wherein the first target-binding domain does not include an immunoglobulin domain.

13. The compound of claim 11 wherein the effector domain is an immunoglobulin effector domain or an non-immunoglobulin effector domain.

14. A method comprising:
providing (i) a first cell that includes a first nucleic acid that encodes a first polypeptide that includes a first immunoglobulin domain and a first interaction sequence, and (ii) a second cell that includes a second nucleic acid encoding a second polypeptide that includes a second interaction sequence and an effector domain;
culturing the first cell under conditions such that the first polypeptide is expressed and the second cell under conditions such that the second polypeptide is expressed;
isolating the first polypeptide from the first cell and the second polypeptide from the second cell; and
contacting the first polypeptide to the second polypeptide to form a complex.

15. The method of claim 14 wherein the first cell is a bacterial cell.

16. The method of claim 15 wherein the second cell is a eukaryotic cell, and the second polypeptide is glycosylated by the second cell.

17. The method of claim 14 further comprising evaluating the complex for an extracellular activity.

18. The method of claim 14 further comprising contacting the complex to a test cell.

19. The method of claim 16 further comprising evaluating the complex for a cytotoxic activity.

20. The method of claim 19 wherein the cytotoxic activity is antibody dependent cell-mediated cytotoxicity or complement mediated cytotoxicity.


SEQUENCE LISTING (8 sequences)

SEQ ID NO: 1 - protein, 29 aa, Homo sapiens
LQAETDQLEDEKSALQTEIANLLKEKEKL

SEQ ID NO: 2 - protein, 29 aa, Homo sapiens
LEEKVKTLKAQNSELASTANMLREQVAQL

SEQ ID NO: 3 - protein, 38 aa, Homo sapiens
LTDTLQAETDQLEDEKSALQTEIANLLKEKEKLEFILA

SEQ ID NO: 4 - protein, 39 aa, Homo sapiens
RIARLEEKVKTLKAQNSELASTANMLREQVAQLKQKVMN

SEQ ID NO: 5 - protein, 29 aa, Homo sapiens
LEEKVKTLKAQNSELASTFNMLREQFAQL

SEQ ID NO: 6 - protein, 29 aa, Homo sapiens
LEEKVKTLKAQNSELASTANMLREQVAQF

SEQ ID NO: 7 - protein, 29 aa, Homo sapiens
LEEKVKTFKAQNSELASTANMLREQVAQF

SEQ ID NO: 8 - protein, 29 aa, Homo sapiens
LEEKVKSFKAQNSEHASTANMLREQVAQL